SEQUENCE LISTING (11 sequences)

SEQ ID NO: 1 - DNA, 2610 bp, Homo sapiens
atcctgggacagggcacagggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggaacgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcagcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctacagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattct

SEQ ID NO: 2 - protein, 480 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

SEQ ID NO: 3 - DNA, 1715 bp, Homo sapiens
gaattccagcggcggcgccgttgccgctgccgggaaacacaaggaaagggaaccagcgcagcgtggcgatgggcgggggtagagccccgccggagaggctgggcggctgccggtgacagactgtgccctgtccacggtgcctcctgcatgtcctgctgccctgagctgtcccgagctaggtgacagcgtaccacgctgccaccatgaatgaggtgtctgtcatcaaagaaggctggctccacaagcgtggtgaatacatcaagacctggaggccacggtacttcctgctgaagagcgacggctccttcattgggtacaaggagaggcccgaggcccctgatcagactctaccccccttaaacaacttctccgtagcagaatgccagctgatgaagaccgagaggccgcgacccaacacctttgtcatacgctgcctgcagtggaccacagtcatcgagaggaccttccacgtggattctccagacgagagggaggagtggatgcgggccatccagatggtcgccaacagcctcaagcagcgggccccaggcgaggaccccatggactacaagtgtggctcccccagtgactcctccacgactgaggagatggaagtggcggtcagcaaggcacgggctaaagtgaccatgaatgacttcgactatctcaaactccttggcaagggaacctttggcaaagtcatcctggtgcgggagaaggccactggccgctactacgccatgaagatcctgcgaaaggaagtcatcattgccaaggatgaagtcgctcacacagtcaccgagagccgggtcctccagaacaccaggcacccgttcctcactgcgctgaagtatgccttccagacccacgaccgcctgtgctttgtgatggagtatgccaacgggggtgagctgttcttccacctgtcccgggagcgtgtcttcacagaggagcgggcccggttttatggtgcagagattgtctcggctcttgagtacttgcactcgcgggacgtggtataccgcgacatcaagctggaaaacctcatgctggacaaagatggccacatcaagatcactgactttggcctctgcaaagagggcatcagtgacggggccaccatgaaaaccttctgtgggaccccggagtacctggcgcctgaggtgctggaggacaatgactatggccgggccgtggactggtgggggctgggtgtggtcatgtacgagatgatgtgcggccgcctgcccttctacaaccaggaccacgagcgcctcttcgagctcatcctcatggaagagatccgcttcccgcgcacgctcagccccgaggccaagtccctgcttgctgggctgcttaagaaggaccccaagcagaggcttggtggggggcccagcgatgccaaggaggtcatggagcacaggttcttcctcagcatcaactggcaggacgtggtccagaagaagctcctgccacccttcaaacctcaggtcacgtccgaggtcgacacaaggtacttcgatgatgaatttaccgcccagtccatcacaatcacaccccctgaccgctatgacagcctgggcttactggagctggaccagcggacccacttcccccagttctcctactcggccagcatccgcgagtgagcagtctgcccacgcagaggacgcacgctcgctgccatcaccgctgggtggttttttacccctgcc

SEQ ID NO: 4 - protein, 481 aa, Homo sapiens
MNEVSVIKEGWLHKRGEYIKTWRPRYFLLKSDGSFIGYKERPEAPDQTLPPLNNFSVAECQLMKTERPRPNTFVIRCLQWTTVIERTFHVDSPDEREEWMRAIQMVANSLKQRAPGEDPMDYKCGSPSDSSTTEEMEVAVSKARAKVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIRE

SEQ ID NO: 5 - DNA, 1547 bp, Homo sapiens
gggagtcatcatgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatatgatgaggatggtatggactgcatggacaatgagaggcggccgcatttccctcaattttcctactctgcaagtggacgagaataagtctctttcattctgctacttcactgtcatcttcaatttattactgaaaatgattcctggacatcaccagtcctagctcttacacatagcaggggca

SEQ ID NO: 6 - protein, 479 aa, Homo sapiens
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDEDGMDCMDNERRPHFPQFSYSASGRE

SEQ ID NO: 7 - DNA, 1695 bp, Homo sapiens
gaattccccaaaccctaaagctgatatcacaaagtaccatttctccaagttgggggctcagaggggagtcatcatgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgctggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatgtcagcaatcagattgtggcatgctgggtaactggaaaaaataataaaaatcggcttcctacagccagcagcacagtcacccatggaactgttggctttggattaaatgtggaattgaacgactacccagaagtgttctggaaagaagcgagatgtgtggcctgcctcaccgtcctcacccatcaaaagcaccagcaggcacgttaactcgaattctcacaaggaaaaggccattaaagctcaaggtgcatttcaaactccaggctac

SEQ ID NO: 8 - protein, 465 aa, Homo sapiens
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKCQQSDCGMLGNWKK

SEQ ID NO: 9 - DNA, 1212 bp, Homo sapiens
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 10 - protein, 403 aa, Homo sapiens [sequence omitted]

SEQ ID NO: 11 - protein, 20 aa, Homo sapiens
CGPKGPGRRGRRRTSSFAEG

(19) United States
(12) Patent Application Publication
(10) Pub. No.: US 2006/0241108 A1
(43) Pub. Date: Oct. 26, 2006

(54) Substituted phenoxazines and acridones as inhibitors of AKT

(75) Inventors: Peter J. Houghton, Memphis, TN (US); Kuntebommenahalli N. Thimmaiah, Memphis, TN (US); John B. Easton, Horn Lake, MS (US)

Correspondence Address: DARBY & DARBY, P.O. BOX 5257, NEW YORK, NY, 10150-5257, US

(73) Assignee: St. Jude Children's Research Hospital, Memphis, TN (US)

(21) Appl. No.: 11/367,161

(22) Filed: Mar. 3, 2006

Related U.S. Application Data
(60) Provisional application No. 60/658,852, filed on Mar. 3, 2005.

(51) Int. Cl. A61K 31/538

(52) U.S. Cl. 514/229.8

(57) ABSTRACT

The invention provides compositions and methods that modulate the activity of AKT family kinase proteins, including AKT1, AKT2 and AKT3 (also referred to as PKBα, PKBβand PKBγ). Specifically, the invention provides a number of phenoxazine and acridone compounds that inhibit AKT phosphorylation and kinase activity. The invention provides compositions for and methods of modulating AKT activity, inhibiting cell growth, treating cancer, treating transplant rejection, and treating coronary artery disease based upon the phenoxazine and acridone compounds of the invention.

### 1. STATEMENT REGARDING FEDERALLY SPONSORED RESEARCH OR DEVELOPMENT

Research or development leading to this invention was supported, at least in part, by Awards CA23099, CA96996 and CA77776 from the United States Public Health Service (USPHS), and by Award CA21675 (Cancer Center Support Grant) from the National Cancer Institute. The United States government may have certain rights to this invention pursuant to the terms of these awards.

### 2. FIELD OF THE INVENTION

The invention provides compositions and methods that modulate the activity of AKT family kinase proteins, including AKT1, AKT2 and AKT3 (also referred to as PKBα, PKBβ and PKBγ). Specifically, the invention provides a number of phenoxazine and acridone compounds that inhibit AKT phosphorylation and kinase activity. The invention provides compositions for and methods of modulating AKT activity, inhibiting cell growth, treating cancer, treating transplant rejection, and treating coronary artery disease based upon the phenoxazine and acridone compounds of the invention.

### 3. BACKGROUND OF THE INVENTION

The AKT family of proteins represents a subfamily of the AGC (protein A, protein G, protein C) family of kinases whose individual members are serine/threonine kinases. The AKT subfamily is also referred to as protein kinase B (PKB). AKT orthologs have been identified in a variety of species, including human (see, e.g., Staal Proc Natl Acad Sci USA 1987;84:5034-5037 and Nakatani et al. J. Biol. Chem. 1999;274:21528-21532), mouse (see, e.g., Yang et al. J. Biol Chem 2003;278:32124-32131), chicken (see, e.g., GenBank Accession number AAB94767), zebrafish (see, e.g., Chan et. al. Cancer Cell 2002;1:257-267), Xenopus (see, e.g., GenBank Accession number AAG59601), Drosophila melanogaster (see, e.g., Franke et al. Oncogene 1994;9: 141-148), Caenorhabditis elegans (see, e.g., Paradis and Ruvkun. Genes & Dev 1998;12:2488-2498), Hydra (see, e.g., Herold et al. Dev Genes Evol 2002;212;513-519), and Anopheles (see, e.g., GenBank Accession number AU06122). In mammalian cells, the AKT subfamily comprises at least three major isoforms that are referred to here as AKT1 (also known as PKBα or RAC-PKα), AKT2 (also known as PKBβ or RAC-PKβ), and AKT3 (also known as PKBγ or RAC-PKγ). An alignment of exemplary amino acid sequences for human AKT 1 (SEQ ID NO: 2), human AKT2 (SEQ ID NO: 4), and two variants of human AKT3 (SEQ ID NOs 6 and 8) are shown in FIG. 1.

Generally speaking, the individual members of the AKT family are highly conserved proteins having at least 85% sequence identity to each other. AKT family proteins contain an N-terminal pleckstrin homology domain, which mediates lipid-protein and protein-protein interactions; a short α-helical linker region; a central serine/threonine kinase domain; and a C-terminal hydrophobic and proline-rich domain (Datta et al. Genes Dev. 1999, 13:2905-2927). For example, in the case of the amino acid sequence of human AKT1 (SEQ ID NO: 2); amino acids 6-107 form the pleckstrin homology domain, amino acids 149-408 form the serine/threonine kinase domain, and amino acids 423-427 form the proline rich domain.

The AKT kinases are associated with a variety of physiological responses, including the inhibition of apoptosis and promotion of cell survival (see, e.g., Kandel & Hay Exp. Cell. Res. 1999;253:210-229). Extensive evidence has also demonstrated a crucial role for AKT in tumorigenesis (see, e.g., Testa & Bellacosa Proc. Natl. Acad. Sci. USA 2001;98: 10983-10985 and Datta et al. Genes Dev. 1999; 13:2905-2927). Furthermore, activation of AKT has been shown to associate with tumor invasiveness and chemoresistance (see, e.g., West et al. Drug Resist Update. 2002;5:234-248). AKT is overexpressed in gastric adenocarcinoma (see, e.g., Staal. Proc. Natl. Acad. Sci. USA 1997;84:5034-5037), breast cancer (see, e.g., Bellacosa et al. Int. J. Cancer 1995;64:280-285), ovarian cancer (see, e.g., Thompson et al. Cancer Genet. Cytogenet. 1996;87:55-62), pancreatic cancer (see, e.g., Cheng et al. Proc. Natl. Acad. Sci. USA 1996;93:3636-3641), and in both estrogen receptor-deficient breast cancer and androgen-independent prostate cell lines (see, e.g., Nakatani et al. J. Biol. Chem. 1999;274:21528-21532). AKT is also activated by the BCR/ABL fusion gene in chronic myelogenous leukemia (see, e.g., Thompson and Thompson. J Clin Oncol 2004;22:4217-26.

The serine/threonine protein kinase AKT is a downstream target of phosphatidylinositol 3-kinase (PI 3-kinase or PI 3-K) (Testa & Bellacosa Proc. Natl. Acad. Sci. USA 2001;98:10983-10985 and Coffer et al. J. Biochem. 1998;335:1-13). PI 3-kinase itself phosphorylates the D-3-hydroxyl position of the myo-inositol ring of phosphatidylinositol (PtdIns) (Stephens et al. Curr. Biol. 1994;4:203-213) to generate the PtdIns-3-phosphates, PtdIns(3)P, PtdIns(3,4)P2(PIP2) and PtdIns(3,4,5)P3(PIP3) (Vanhaesebroeck et al. Trends Biochem. Sci. 1997;275: 1848-1850). PI 3-kinase-generated phospholipids activate AKT activity by multiple mechanisms, including direct binding of phosphoinositides to the pleckstrin homology domain of AKT and translocation of AKT from the cytoplasm to the nucleus (Datta et al. Genes & Dev 1999; 13:2905-2927). PI 3-kinase is activated by many growth factor receptors and oncogenic protein tyrosine kinases (Cantley et al. Cell 1991;64:281-302; Stephens et al. Biochim. Biophys. Acta 1993;1179:27-75; and Varticovski et al. Biophys. Acta, 1994;1226:1-11) as well as by p21Ras (Mcllroy et al. Mol. Cell. Biol. 1997;17:248-255), leading to increased cell growth and inhibition of apoptosis (Kapeller et al. Bioessays 1994; 16:565-576 and Yu et al. Biol. Chem. 1998;273:30199-30203). PI 3-kinase expression is increased in ovarian cancer (see, e.g., Shayesteh et al. Nat. Genet. 1999;21:99-102), breast cancer (see, e.g., Salh et al. Int J Cancer 2002;98:148-154), and epithelial carcinoma of the mouth (see, e.g., Stahl et al. Pathologe 2004;25:31-7). Genetic amplification of PI 3-kinase has been reported for ovarian cancer (see, e.g., Gao et al. Am J Physiol Cell Physiol 2004;287:C281-C291), lung cancer (see, e.g., Massion et al. Am J Respir Crit Care Med 2004; 170:1088-1094), gastric carcinoma (see, e.g., Byun et al. Int J Cancer 2003;104:318-327), cervical cancer (see, e.g., Ma et al. Oncogene 2000; 19:2739-2744), and glioblastoma (see, e.g., Knobbe and Reifenberger. Brain Pathol 2003;13:507-518). PI 3-kinase is constitutively activated in human small cell lung cancer cell lines, where it leads to anchorage-independent growth and has been suggested to be a cause of metastasis (see, e.g., Moore et al. Cancer Res. 1998;58:5239-5247). The major role for PI 3-kinase in cancer cell growth is its role in survival signaling mediated by AKT to prevent apoptosis (Krasilnikov Biochemistry (Mosc.) 2000;65:59-67).

Activation of AKT is negatively regulated by the tumor suppressor protein phosphatase and tensin homolog deleted on chromosome 10 (PTEN), a tyrosine-threonine/lipid phosphatase that dephosphorylates the 3-position of PtdIns-3-phosphate (Wu et al. Proc. Natl. Acad. Sci. USA 1998;95:15587-15591 and Maehama et al. J. Biol. Chem. 1998;273:13375-13378) A broad variety of human cancers harbor PTEN alterations, including glioblastomas and endometrial, breast, thyroid, and prostate cancers (see, e.g., Wu et al. Oncogene 2003;22:3113-3122 and Steck et al. Nat Genet 1997; 15:356-362) as well as cervical cancer (see, e.g., Minaguchi et al. Cancer Lett 2004;210:57-62). Alterations in the level of PTEN activity have been identified in colorectal cancer (see, e.g., Goel et al. Cancer Res. 2004;64:3014-3021 and Nassifet al. Oncogene 2004;23:617-628), lung cancer (see, e.g., Goncharuk et al. Ann Diagn Pathol. 2004;8:6-16), gastric cancer (see, e.g., Kang et al. Lab Invest. 2002;82:285-291). Mutations in PTEN are also causative for two related human hereditary cancer predisposition syndromes: Cowden Disease and Bannayan-Zonana syndrome (see, e.g., Sansal and Sellers. J Clin Oncol 2004;22:2954). Mutations in PTEN which lead to activation of AKT pathway have been identified in various tumors (see, e.g., Cheng et al. In: Schwab, ed. Encyclopedic Reference of Cancer. Berlin, Germany: Springer: 2001).

The design and development of small molecules that specifically inhibit the kinase activity of AKT and the AKT signal transduction pathway is therefore an attractive approach for the development of new therapeutic agents, e.g., for cancer. A number of publications describe testing various compounds for their ability to inhibit both PI 3-kinase and AKT activities. These compounds include phosphatidylinositol (PI) analogues (Hu et al. J. Med. Chem. 2000;43:3045-3051; Hu et al. Bioorg. Med. Chem. Lett. 2001;11: 173-176; Kozikowski et al. Chem. Soc. 2003;125:1144-1145; and Meuillet et al. Mol. Cancer Therapeut. 2003;2:389-399), H-89 analogues (Reuveni et al. Biochemistry 2002;41:1034-10314), azapane derivatives (Breitenlechner et al. J. Med. Chem. 2004;47:1375-1390), peptide inhibitors (Luo et al. Biochemistry 2004;43: 1254-1263), the small molecule Akt pathway inhibitor known as Akt/protein kinase B signaling inhibitor-2 (API-2, also known as triciribine or TCN, NCI Diversity set identifier NSC 154020) (Yang et al. Cancer Res. 2004;64:4394-4399) and compounds containing planar aromatic heterocycles (Kau et al. Cancer Cell. 2003;4:463-476), including phenothiazine derivatives such as trifluroperazine. Isozyme selective inhibitors of AKT have also been reported.

The chemistry and biology of N¹⁰-substituted phenoxazines, which were synthesized originally as modulators of P-glycoprotein mediated multidrug resistance (MDR), has been described (see Thimmaiah et al. Cancer Commun. 1990;2:249-259; Thimmaiah et al. J. Med. Chem. 1992;35:3358-3364; Horton et al. Mol. Pharmacol. 1993;44:552-559; Eregowda et al. Indian J. Chem. 2000;39B:243-259; and Houghton et al. U.S. Pat. No. 5,371,081). However, several of the N¹⁰-substituted phenoxazine compounds are reported to enhance vincristine toxicity in cells with undetectable levels of P-glycoprotein. This has lead to the suggestion that at least part of the activity of some phenoxazine-based MDR modulators might be mediated through a P-glycoprotein-independent mechanism. However, the exact mechanism has not been identified and remains unknown.

The chemistry and biology of 2-methoxy-N¹⁰-substituted acridones (Krishnegowda et al. Bioorg Med Chem 2002;10:2367-2380) and 4-unsubstituted and 4-methoxy acridones (Hegde et al. Eur J Med Chem 2004;39:161-177), which were synthesized originally as modulators of P-glycoprotein mediated multidrug resistance (MDR), have been described. However, the exact mechanism has not been identified and remains unknown.

The design and development of small molecules that specifically inhibit the activity of AKT and the AKT signal transduction pathway is an attractive approach for the development of new therapeutic agents, e.g., for cancer. Hence, there is an ongoing and unmet need for compositions and methods of modulating AKT activity in cells.

The citation and/or discussion of a reference in this section and throughout the specification is provided merely to clarify the description of the present invention and is not an admission that any such reference is “prior art” to the invention described herein.

### 4. BRIEF SUMMARY OF THE INVENTION

The invention is directed to phenoxazine compounds. In particular, the invention provides phenoxazine compounds of Formula (I): 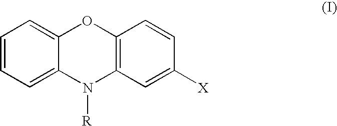
and pharmaceutically acceptable salts thereof,
wherein

X is selected from hydrogen, halogen, and haloalkyl;

R is selected from hydrogen and (CH₂)ₙA;

wherein

n is an integer selected from 2, 3, 4, 5, and 6; and

A is selected from —NR₁R₂;

wherein

R₁ and R₂ are independently selected from hydrogen, linear or branched alkyl, linear or branched alkyl substituted with one or more hydroxyl groups, phenyl, and substituted phenyl; or

R₁ and R₂ when taken together with the nitrogen atom to which they are attached, optionally form a cyclic ring of the formula (II): 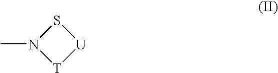
wherein

S and T are independently alkylene having 1, 2, 3, or 4 carbon atoms; and

U is selected from —O—, —S—, —N(R₃)—, and —CH(R₄)—;

wherein

R₃ and R₄ are independently selected from hydrogen, linear or branched alkyl, and linear or branched alkyl substituted with one or more hydroxyl groups. In preferred embodiments, S and T are independently alkylene having 1, 2, 3, or 4 carbon atoms; and U is selected from —O—, —S—, —N(R₃)—, and —CH(R₄)—; with the proviso that when S and T are both —(CH₂)₂—, U is not —O—.

In preferred embodiments, n is 3 or 4. In particularly preferred embodiments, n is 4.

In preferred embodiments, R₁ and R₂ are independently selected from ethyl, n-propyl, co-hydroxyethyl and co-hydroxypropyl.

In preferred embodiments, the phenoxazine compound of Formula (I) is selected from:
2-chlorophenoxazine,
10-[3′-(N-diethylamino)-propyl]-2-chlorophenoxazine,
10-[3′-[N-bis(hydroxyethyl) amino] propyl]-2-chlorophenoxazine,
10-(3′-N-piperidinopropyl)-2-chlorophenoxazine,
10-(3′-N-pyrrolidinopropyl)-2-chlorophenoxazine,
10-[3′-[(β-hydroxyethyl) piperazino]propyl]-2-chlorophenoxazine,
10-[4′-(N-diethylamino)butyl]-2-chlorophenoxazine,
10-[4′-[N-bis(hydroxyethyl) amino]butyl]-2-chlorophenoxazine,
10-(4′-N-piperidinobutyl)-2-chlorophenoxazine,
10-(4′-N-pyrrolidinobutyl)-2-chlorophenoxazine,
10-[4′-[(β-hydroxyethyl) piperazino]butyl]-2-chlorophenoxazine,
10-[4′-[N-bis(hydroxyethyl) amino]butyl]-2-trifluoromethyl phenoxazine,
10-(4′-N-piperidinobutyl)-2-trifluoromethylphenoxazine,
10-[3′-[N-bis(hydroxyethyl) amino]propyl] phenoxazine,
10-(3′-N-pyrrolidinopropyl)-phenoxazine,
10-[4′-[N-bis(hydroxyethyl) amino]-butyl]phenoxazine,
10-(4′-N-pyrrolidinobutyl)-phenoxazine,
10-[4′-[(β-hydroxyethyl piperazino]butyl]-phenoxazine, and
10-(3′-N-benzylaminopropyl)-phenoxazine.
   and pharmaceutically acceptable salts thereof.

In particularly preferred embodiments, the phenoxazine compound of Formula (I) is selected from:
10-[4′-(N-diethylamino)butyl]-2-chlorophenoxazine, and
10-[4′-[(β-hydroxyethyl) piperazino]butyl]-2-chlorophenoxazine.
   and pharmaceutically acceptable salts thereof.

The invention is also directed to acridone compounds. In particular, the invention provides acridone compounds of Formula (III): 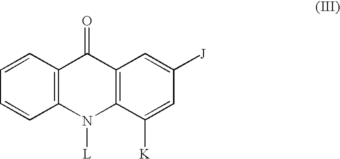
and pharmaceutically acceptable salts thereof,
wherein

J is selected from hydrogen, halogen, or alkoxy;

K is selected from hydrogen or alkoxy; and

L is selected from hydrogen and (CH₂)_{nB};

wherein

n is an integer selected from 2, 3, 4, 5, and 6; and

B is selected from halogen and —NR₅R₆;

wherein

R₅ and R₆ are independently selected from hydrogen, linear or branched alkyl, linear or branched alkyl optionally substituted with one or more hydroxyl groups; or

R₅ and R₆ when taken together with the nitrogen atom to which they are attached, optionally form a cyclic ring of the formula (IV): 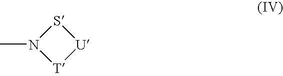
wherein

S′ and T′ are independently alkylene having 1, 2, 3, or 4 carbon atoms; and

U′ is selected from —O—, —S—, —N(R₇)—, and —CH(R₈)—;

wherein

R₇ and R₈ are independently selected from hydrogen, linear or branched alkyl, and linear or branched alkyl substituted with one or more hydroxyl groups.

In preferred embodiments, J is selected from hydrogen, Cl, Br, and OCH₃, and K is selected from hydrogen and OCH₃.

In preferred embodiments, the acridone compound of formula (III) is selected from:
10-(3′-N-Diethylaminopropyl)-2-chloroacridone
10-[3′-N-(Methylpiperazino)propyl]-2-chloroacridone
10-(3′-N-Piperidinopropyl)-2-chloroacridone
10-[3′-N-Pyrrolidinopropyl]-2-chloroacridone
10-(3′-N-Morpholinopropyl)-2-chloroacridone
10-(3′-Chloropropyl)-2-chloroacridone
10-(4′-N-Diethylaminobutyl)-2-chloroacridone
10-(4′-N-(Methylpiperazino) butyl)-2-chloroacridone
10-(4′-N-Piperidinobutyl)-2-chloroacridone
10-(4′-N-[(β-Hydroxyethyl)piperazino]butyl)-2-chloroacridone
10-[4′-N-Pyrrolidinobutyl]-2-chloroacridone
10-(4′-N-Morpholinobutyl)-2-chloroacridone
10-(4′-Chlorobutyl)-2-chloroacridone
10-(4′-N-Piperidinobutyl)-2-methoxyacridone
10-(4′-N-([β-Hydroxyethyl]piperazino)butyl)-2-bromoacridone
10-(3′-N-[(β-Hydroxyethyl) piperazino] propyl)-2-bromoacridone
10-(3′-N-[Bis[hydroxyethyl]amino]propyl)-2-bromoacridone
10-(4′-N-Chlorobutyl)-2-bromoacridone
10-(3′-N-Morpholinopropyl)-2-bromoacridone
10-(4′-[N-Diethylamino)butyl)-2-bromoacridone
10-(4′-N-Pyrrolidinobutyl)-2-bromoacridone
10-(4′-N-Morpholinobutyl)-2-bromoacridone
10-(3′-N-Piperidinopropyl)-2-bromoacridone
10-(4′-N-Thiomorpholinobutyl)-2-bromoacridone
10-(3′-N-Pyrrolidinopropyl)-2-bromoacridone
10-(3′-[N-Diethylamino]propyl)-2-bromoacridone
   and pharmaceutically acceptable salts thereof.

In particularly preferred embodiments, the acridone compound of formula (III) is selected from:
10-[3′-N-(Methylpiperazino)propyl]-2-chloroacridone,
10-(3′-Chloropropyl)-2-chloroacridone,
10-(4′-N-Diethylaminobutyl)-2-chloroacridone,
10-(4′-N-(Methylpiperazino) butyl)-2-chloroacridone,
10-(4′-N-Piperidinobutyl)-2-chloroacridone,
10-(4′-N-[(β-Hydroxyethyl)piperazino]butyl)-2-chloroacridone,
10-(4′-Chlorobutyl)-2-chloroacridone,
10-(4′-N-Pyrrolidinobutyl)-2-bromoacridone,
10-(4′-N-Morpholinobutyl)-2-bromoacridone,
10-(3′-N-Pyrrolidinopropyl)-2-bromoacridone, and
10-(3′-[N-Diethylamino]propyl)-2-bromoacridone.
   and pharmaceutically acceptable salts thereof.

The invention is also directed to a method of modulating AKT activity, said method comprising contacting an AKT with an effective amount of a phenoxazine compound or an acridone compound, or pharmaceutically acceptable salts thereof. In a particular embodiment the phenoxazine compounds and acridone compounds are the compounds of Formula (I) and Formula (III) or pharmaceutically acceptable salts thereof, respectively. In preferred embodiments, contacting an AKT comprises contacting a cell comprising an AKT. In particularly preferred embodiments, the cell is a mammalian cell.

The invention is further directed to a method of inhibiting cell growth of a cell, said method comprising contacting the cell with an effective amount of a phenoxazine compound or an acridone compound, or pharmaceutically acceptable salts thereof. In a particular embodiment the phenoxazine compounds and acridone compounds are the compounds of Formula (I) and Formula (III) or pharmaceutically acceptable salts thereof, respectively. In preferred embodiments, the cell is a mammalian cell. The invention is also directed to a method of inhibiting cell growth of a cell, wherein the cell is a cell in which AKT is activated, said method comprising contacting the cell with an effective amount of a phenoxazine compound or an acridone compound, or pharmaceutically acceptable salts thereof. In a particular embodiment the phenoxazine compounds and acridone compounds are the compounds of Formula (I) and Formula (III) or pharmaceutically acceptable salts thereof, respectively. In preferred embodiments, the cell is a mammalian cell.

The invention is further directed to a method of treating cancer in a patient, said method comprising administering to a patient in need of such treatment an effective amount of a phenoxazine compound or acridone compound, or pharmaceutically acceptable salts thereof. In a particular embodiment the phenoxazine compounds and acridone compounds are the compounds of Formula (I) and Formula (III) or pharmaceutically acceptable salts thereof, respectively. In preferred embodiments, the patient is a mammal. In particularly preferred embodiments, the patient is a human.

The invention is further directed to a method of treating cancer in a patient, wherein the cancer is a cancer in which AKT is activated, said method comprising administering to a patient in need of such treatment an effective amount of a phenoxazine compound or an acridone compound, or pharmaceutically acceptable salts thereof. In a particular embodiment the phenoxazine compounds and acridone compounds are the compounds of Formula (I) and Formula (III) or pharmaceutically acceptable salts thereof, respectively. In preferred embodiments, the cancer is gastric cancer, breast cancer, ovarian cancer, pancreatic cancer, prostate cancer, chronic myelogenous leukemia, glioblastoma, endometrial cancer, thyroid cancer, cervical cancer, colorectal cancer, lung cancer, or epithelial carcinoma of the mouth. In preferred embodiments, the patient is a mammal. In particularly preferred embodiments, the patient is a human.

The invention is also directed to a method of treating transplant rejection in a patient, said method comprising administering to a patient in need of such treatment an effective amount of a phenoxazine compound or an acridone compound, or pharmaceutically acceptable salts thereof. In a particular embodiment the phenoxazine compounds and acridone compounds are the compounds of Formula (I) and Formula (III) or pharmaceutically acceptable salts thereof, respectively. In preferred embodiments, the patient is a mammal. In particularly preferred embodiments, the patient is a human.

The invention is also directed to a method of treating coronary artery disease, said method comprising administering to a patient in need of such treatment a drug-eluting stent comprising an effective amount of a phenoxazine compound or an acridone compound, or pharmaceutically acceptable salts thereof, in a particular embodiment the phenoxazine compounds and acridone compounds are the compounds of Formula (I) and Formula (III) or pharmaceutically acceptable salts thereof, respectively, wherein the administering comprises placing the drug-eluting stent into the luminal space of at least one coronary artery of the patient. In preferred embodiments, the patient is a mammal. In particularly preferred embodiments, the patient is a human.

The invention is further directed to a drug eluting stent comprising a phenoxazine compound or an acridone compound, or pharmaceutically acceptable salts thereof. In a particular embodiment the phenoxazine compounds and acridone compounds are the compounds of Formula (I) and Formula (III) or pharmaceutically acceptable salts thereof, respectively.

### 5. BRIEF DESCRIPTION OF THE DRAWINGS

FIG. 1 is an alignment of exemplary amino acid sequences for hAKT1 (SEQ IN NO: 2), hAKT2 (SEQ ID NO: 4), hAKT3 isoform variant 1 (“hAKT3 v1”, SEQ ID NO 6), and hAKT isoform variant 2 (“hAKT3 v2”, SEQ ID NO: 8). “*”=the residues in that column are identical in all sequences in the alignment. “:”=conserved substitutions have been observed. “.”=semi-conserved substitutions are observed.

### 6. DETAILED DESCRIPTION OF THE INVENTION

As described in more detail below, the invention provides compositions that modulate the activity of AKT family kinase proteins. Specifically, the invention provides a number of phenoxazine and acridone compounds that inhibit AKT phosphorylation and kinase activity. The invention provides compositions for and methods of modulating AKT activity, inhibiting cell growth, treating cancer, treating transplant rejection, and treating coronary artery disease based upon the phenoxazine and acridone compounds of the invention.

As used herein, the term “AKT” refers any member of the AKT subfamily of the AGC (protein A, protein G, protein C) family of kinases whose individual members are serine/threonine kinases. The nucleotide and amino acid sequences for AKT orthologs from a variety of species (including human, mouse, chicken, zebrafish, Xenopus, Drosophila melanogaster, Caenorhabditis elegans, Hydra, and Anopheles) are known in the art. Generally speaking, the individual members of the AKT family are highly conserved proteins having at least 85% sequence identity to each other. AKT family proteins contain an N-terminal pleckstrin homology domain, which mediates lipid-protein and protein-protein interaction; a short α-helical linker region; a central serine/threonine kinase domain; and a C-terminal hydrophobic and proline-rich domain.

In preferred embodiments, the AKT is AKT1, AKT2, or AKT3. In particularly preferred embodiments the AKT is a mammalian AKT (e.g., mammalian AKT1, mammalian AKT2, or mammalian AKT3). In particularly preferred embodiments, the AKT is a human AKT (HAKT) (e.g. hAKT1, hAKT2, or hAKT3).

Amino acid and nucleotide sequences for AKT1 (also known as PKBα or RAC-PKα) have been reported for a variety of species, including human, mouse, rat, cow, chicken, and Xenopus. In preferred embodiments, AKT1 is a mammalian AKT1. In particularly preferred embodiments, AKT1 is human AKT1 (hAKT1). Exemplary nucleotide and amino acid sequences for human AKT1 are set forth in SEQ ID NOs 1 and 2, respectively.

Amino acid and nucleotide sequences for AKT2 (also known as PKBβ or RAC-PKβ) have been reported for a variety of species, including human, mouse, rat, dog, chicken, Xenopus, and zebrafish. In preferred embodiments, AKT2 is a mammalian AKT2. In particularly preferred embodiments, AKT2 is human AKT2 (hAKT2). Exemplary nucleotide and amino acid sequences for human AKT2 are set forth in SEQ ID NOs 3 and 4, respectively.

Amino acid and nucleotide sequences for AKT3 (also known as PKBγ or RAC-PKγ) have been reported for a variety of species, including human, mouse, rat, dog, and chicken. In the case of human AKT3 alternative splicing results in the production of at least two different hAKT3 isoforms, whose amino acid sequences vary at the C-terminus of the hAKT3 protein. Exemplary nucleotide and amino acid sequences for human AKT3, isoform variant 1, are set forth in SEQ ID NOs 5 and 6, respectively. Exemplary nucleotide and amino acid sequences for human AKT3, isoform variant 2, are set forth in SEQ ID NOs 7 and 8, respectively.

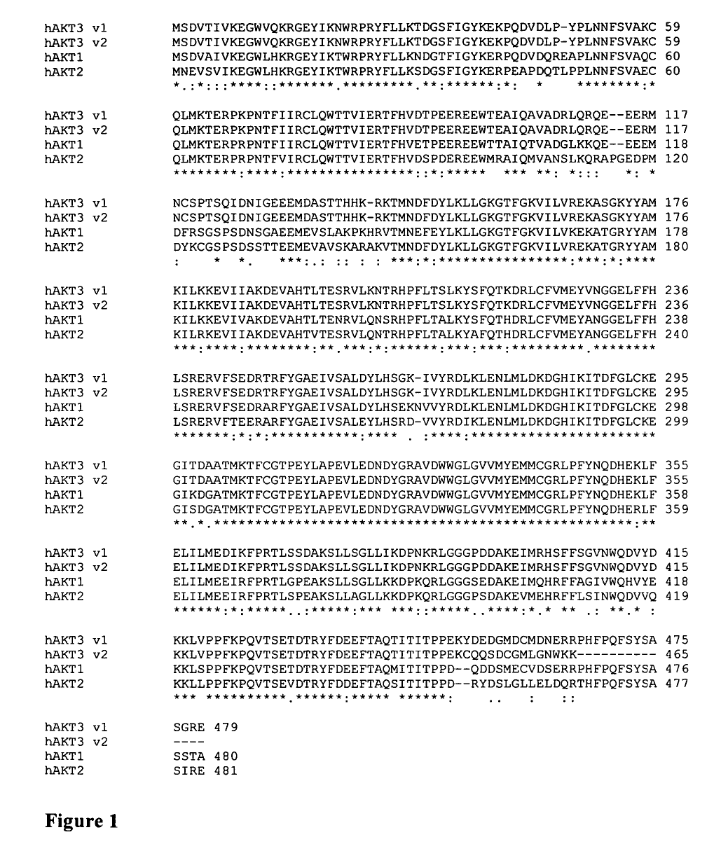

The amino acid sequences for hAKT 1 (SEQ IN NO: 2), hAKT2 (SEQ ID NO: 4), hAKT3 isoform variant 1 (SEQ ID NO 6), and HAKT isoform variant 2 (SEQ ID NO: 8) are shown in FIG. 1.

6.1. AKT Modulating Compounds

The present invention provides phenoxazine and acridone compounds that modulate AKT activity. Preferred phenoxazine and acridone compounds of the invention inhibit AKT activation at low (e.g., micromolar) concentrations and, in particular, specifically block AKT activation and signaling to downstream targets of AKT such as mammalian target of rapamycin (mTOR), p70 ribosomal protein S6 kinase (p70S6 kinase), and ribosomal protein S6 (rpS6 or S6). Preferred phenoxazine and acridone compounds of the invention do not affect the activity of upstream kinases, such as phosphoinositide 3 phosphate dependent kinase 1 (PDK1) or PI 3-kinase. Preferred phenoxazine and acridone compounds of the invention do not affect other kinase pathways downstream of ras, such as the extracellular regulated kinase 1/2 (ERK-1/2) pathway. Preferred compounds of the invention inhibit cell growth and induce apoptosis in cancer cells, such as rhabdomyosarcoma (Rh) cells.

As used herein, the terms “halo” or “halogen” refer to fluoride, chloride, bromide or iodide atoms.

As used herein, the term “alkyl”, alone or in combination, denotes saturated straight or branched chain hydrocarbon radicals having in the range of about one to about twelve carbon atoms. Examples of alkyl include, but are not limited to, methyl, ethyl, n-propyl, isopropyl, n-butyl, sec-butyl, tert-butyl, pentyl, isopentyl, neopentyl, hexyl, isohexyl, heptyl, isoheptyl, and octyl. The term “lower alkyl” denotes straight-chain or branched saturated hydrocarbon residues with one to six carbon atoms, preferably with one to four carbon atoms.

As used herein, the term “haloalkyl” refers to an alkyl radical substituted by one or more halogen atoms. Suitable examples of haloalkyl include, but are not limited to, trifluoromethyl and pentafluoroethyl.

As used herein, the term “alkoxy”, alone or in combination, denotes linear or branched oxy-containing radicals each having alkyl portions of one to about ten carbon atoms. “Lower alkoxy” denotes a lower alkyl group which is bound via an oxygen atom. Examples of such lower alkoxy groups include, but are not limited to, methoxy, ethoxy, n-propoxy, isopropoxy, n-butoxy, and tert-butoxy.

As used herein, the term “substituted phenyl” denotes phenyl radicals wherein at least one hydrogen is replaced by one more substituents such as, but not limited to, hydroxy, alkoxy, halogen, haloalkyl, cyano, nitro, amino, and amido.

Phenoxazine compounds and their derivatives. The compounds of the invention include phenoxazine compounds and derivatives thereof. Preferred compounds of the invention are N¹⁰-substituted phenoxazine compounds (and pharmaceutically acceptable salts thereof) of the general formula (I), below.

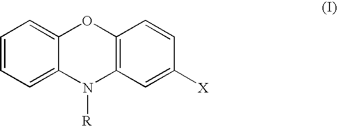

In such compounds:

X is preferably hydrogen, a halogen or a haloalkyl; and

R is preferably a hydrogen or (CH₂)ₙA, wherein

n is an integer having the value 2, 3, 4, 5 or 6; and

A is selected from —NR₁R₂, wherein

R1 and R2 are independently selected from hydrogen, linear or branched alkyl, linear or branched alkyl substituted with one or more hydroxyl groups, phenyl and substituted phenyl; or, alternatively,

R₁ and R₂, taken together with the nitrogen atom to which they are attached optionally form a cyclic ring of formula (II), below:

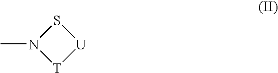

in which S and T are independently selected from alkylenes having 1, 2, 3 or 4 carbon atoms; and

U is selected from —O—, —S—, —N(R₃)— and —CH(R₄), wherein

R₃ and R₄ are independently selected from hydrogen, linear or unbranched alkyl moieties, and linear or unbranched alkyl substituted with one or more hydroxyl groups.

Particularly preferred compounds of the invention are N¹⁰-substituted phenoxazine compounds (and pharmaceutically acceptable salts thereof) of the general formula (I) as described above, wherein S and T are independently selected from alkylenes having 1, 2, 3 or 4 carbon atoms; and U is selected from —O—, —S—, —N(R₃)— and —CH(R₄), with the proviso that when S and T are both —(CH₂)₂—, U is not —O—. However, the invention also encompasses compounds wherein S and T are both —(CH₂)₂— and U is —O—.

In one particularly preferred embodiment of compounds according to formula (I), above, R is (CH₂)ₙNR₁R₂. In such embodiments, particularly preferred values of n are 3 or, even more preferably, 4. In further preferred embodiments, R₁ and R₂ are independently selected from ethyl, n-propyl, co-hydroxyethyl or co-hydroxypropyl.

In other embodiments of compounds according to formula (I), when R is (CH₂)ₙR₁R₂ and NR₁R₂ is represented by formula (II), S and T are each independently —CH₂— or —CH₂—CH₂—. In another preferred embodiment, S and T are both —CH₂—CH₂—, and R₃ and R₄ are independently selected from hydrogen, ethyl, n-propyl, ω-hydroxyethyl or ω-hydroxypropyl. When NR₁R₂ is represented by formula (II), U is preferably N(R₃)— or —CH(R₄). When U is —N(R₃)—, R₃ is preferably CH₂CH₂OH. When U is —CH(R₄)—, R₄ is preferably hydrogen.

In further embodiments, X is preferably selected from hydrogen, Cl and CF₃.

Suitable but non-limiting examples of compounds according to formula (I) are provided, infra, in Table I of the Examples.

Preferred compounds of the invention include:

| Compound | | | |
|---|---|---|---|
| ID* | X | R | Name |
| 1B | Cl | —H | 2-chlorophenoxazine |
| 3B | Cl | —(CH₂)₃—N(CH₂CH₃)₂ | 10-[3′-(N-diethylamino)-propyl]-2-chlorophenoxazine |
| 4B | Cl | —(CH₂)₃—N(CH₂CH₂OH)₂ | 10-[3′-[N-bis(hydroxyethyl)amino]propyl]-2- |
| | | | chlorophenoxazine |
| 6B | Cl | 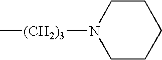 | 10-(3′-N-piperidinopropyl)-2-chlorophenoxazine |
| 7B | Cl | 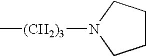 | 10-(3′-N-pyrrolidinopropyl)-2-chlorophenoxazine |
| 8B | Cl | 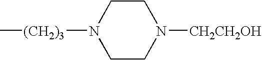 | 10-[3′-[(β-hydroxyethyl)piperazino]propyl]-2- chlorophenoxazine |
| 10B | Cl | —(CH₂)₄—N(CH₂CH₃)₂ | 10-[4′-(N-diethylamino)butyl]-2-chlorophenoxazine |
| 11B | Cl | —(CH₂)₄—N(CH₂CH₂OH)₂ | 10-[4′-[N-bis(hydroxyethyl)amino]butyl]-2-chlorophenoxazine |
| 13B | Cl | 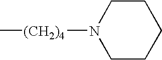 | 10-(4′-N-piperidinobutyl)-2-chlorophenoxazine |
| 14B | Cl | 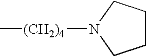 | 10-(4′-N-pyrrolidinobutyl)-2-chlorophenoxazine |
| 15B | Cl | 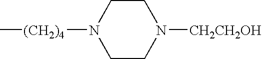 | 10-[4′-[(β-hydroxyethyl)piperazino]butyl]-2- chlorophenoxazine |
| 11C | CF₃ | —(CH₂)₄—N(CH₂CH₂OH)₂ | 10-[4′-[N-bis(hydroxyethyl)amino]butyl]-2-trifluoromethyl |
| | | | phenoxazine |
| 13C | CF₃ | 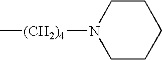 | 10-(4′-N-piperidinobutyl)-2-trifluoromethylphenoxazine |
| 4A | H | —(CH₂)₃—N(CH₂CH₂OH)₂ | 10-[3′-[N-bis(hydroxyethyl)amino]propyl]phenoxazine |
| 8A | H | 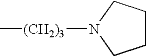 | 10-(3′-N-pyrrolidinopropyl)-phenoxazine |
| 11A | H | —(CH₂)₄—N(CH₂CH₂OH)₂ | 10-[4′-[N-bis(hydroxyethyl)amino]-butyl]phenoxazine |
| 14A | H | 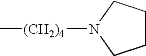 | 10-(4′-N-pyrrolidinobutyl)-phenoxazine |
| 15A | H | 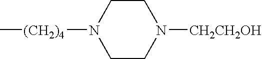 | 10-[4′-[(β-hydroxyethyl piperazino]butyl]-phenoxazine |
| 22A | H | 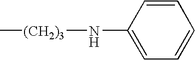 | 10-(3′-N-benzylaminopropyl)-phenoxazine |

Of these, the compounds 10-[4′-(N-diethylamino)butyl]-2-chlorophenoxazine (compound 10B) and 10-[4′-[(P-hydroxyethyl) piperazino]butyl]-2-chlorophenoxazine (compound 15B) are particularly preferred.

Acridone compounds and derivatives thereof. Preferred compounds of the invention also include acridone compounds and derivatives (including pharmaceutically acceptable salts) thereof. Particularly preferred acridone compounds are compounds of formula (III), below.

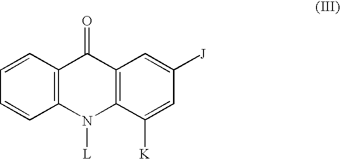

wherein:

J can be hydrogen, a halogen or an alkoxy;

K can be a hydrogen or an alkoxy; and

L can be a hydrogen or (CH₂)ₙB, wherein

n is an integer between 2 and 6 (i.e., n can be 2, 3, 4, 5 or 6); and

B can be a halogen or _{—NR5R6}, wherein

R5 and R6 are independently selected from a halogen, a linear or unbranched alkyl, and a linear or unbranched alkyl optionally substituted with one or more hydroxyl groups.

Alternatively, R₅ and R₆, when taken together with the nitrogen atom to which they are attached, optionally form a cyclic ring of the formula (IV), below.

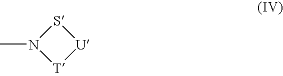

In formula (IV),

S′ and T′ are each independently selected from alkynes having 1,2, 3 or 4 carbon atoms; and

U′ can be —O—, —S—, —N(R₇)—, or —CH(R₈)—, wherein

R7 and R8 are independently selected from hydrogen, linear or branched alkyls, and linear or branched alkyls substituted with one or more hydroxyl moieties.

In preferred embodiments of compounds according to formula (III), above, L is (CH₂)ₙNR₅R₆. In such embodiments, In such embodiments, particularly preferred values of n are 3 or, even more preferably, 4. In further preferred embodiments, R₁ and R₂ are independently selected from ethyl, n-propyl, Ω-hydroxyethyl or Ω-hydroxypropyl.

In other embodiments of compounds according to formula (III), when L is (CH₂)ₙR₅R₆ and NR₅R₆ is represented by formula (IV), S′ and T′ are each independently —CH₂— or —CH₂—CH₂—. In another preferred embodiment, S′ and T′ are both —CH₂—CH₂—, and R₇ and R₈ are independently selected from hydrogen, ethyl, n-propyl, co-hydroxyethyl or co-hydroxypropyl. When NR₅R₆ is represented by formula (IV), U′ is preferably N(R₇)— or —CH(R₈). When U is —N(R₇)—, R₇ is preferably CH₂CH₂OH. When U is —CH(R₈)—, R₈ is preferably hydrogen.

In preferred embodiments of compounds according to formula (III), J is halogen. In further embodiments of compounds according to formula (III), J is preferably selected from hydrogen, Cl, Br and OCH₃. In particularly preferred embodiments, J is Cl or Br. In still other embodiments of compounds according to formula (III), K is preferably selected from hydrogen and OCH₃.

Suitable but non-limiting examples of compounds according to formula (III) are provided, infra, in Table III of the Examples. These include the following compounds:

| Compound | |
|---|---|
| ID | Name |
| 1 | 10-(3′-N-Diethylaminopropyl)-2-chloroacridone |
| 2 | 10-[3′-N-(Methylpiperazino)propyl]-2-chloroacridone |
| 3 | 10-(3′-N-Piperidinopropyl)-2-chloroacridone |
| 4 | 10-[3′-N-Pyrrolidinopropyl]-2-chloroacridone |
| 5 | 10-(3′-N-Morpholinopropyl)-2-chloroacridone |
| 6 | 10-(3′-Chloropropyl)-2-chloroacridone |
| 7 | 10-(4′-N-Diethylaminobutyl)-2-chloroacridone |
| 8 | 10-(4′-N-(Methylpiperazino)butyl)-2-chloroacridone |
| 9 | 10-(4′-N-Piperidinobutyl)-2-chloroacridone |
| 10 | 10-(4′-N-[(β-Hydroxyethyl)piperazino]butyl)-2- |
| | chloroacridone |
| 11 | 10-[4′-N-Pyrrolidinobutyl]-2-chloroacridone |
| 12 | 10-(4′-N-Morpholinobutyl)-2-chloroacridone |
| 13 | 10-(4′-Chlorobutyl)-2-chloroacridone |
| 14 | 10-(4′-N-Piperidinobutyl)-2-methoxyacridone |
| 15 | 10-(4′-N-([β-Hydroxyethyl]piperazino)butyl)-2- |
| | bromoacridone |
| 16 | 10-(3′-N-[(β-Hydroxyethyl) piperazino] propyl)-2- |
| | bromoacridone |
| 17 | 10-(3′-N-[Bis[hydroxyethyl]amino]propyl)-2- |
| | bromoacridone |
| 18 | 10-(4′-N-Chlorobutyl)-2-bromoacridone |
| 19 | 10-(3′-N-Morpholinopropyl)-2-bromoacridone |
| 20 | 10-(4′-[N-Diethylamino)butyl)-2-bromoacridone |
| 21 | 10-(4′-N-Pyrrolidinobutyl)-2-bromoacridone |
| 22 | 10-(4′-N-Morpholinobutyl)-2-bromoacridone |
| 23 | 10-(3′-N-Piperidinopropyl)-2-bromoacridone |
| 24 | 10-(4′-N-Thiomorpholinobutyl)-2-bromoacridone |
| 25 | 10-(3′-N-Pyrrolidinopropyl)-2-bromoacridone |
| 26 | 10-(3′-[N-Diethylamino]propyl)-2-bromoacridone |

Of these, the following compounds are particularly preferred:

| Compound | |
|---|---|
| ID | Name |
| 2 | 10-[3′-N-(Methylpiperazino)propyl]-2-chloroacridone |
| 6 | 10-(3′-Chloropropyl)-2-chloroacridone |
| 7 | 10-(4′-N-Diethylaminobutyl)-2-chloroacridone |
| 8 | 10-(4′-N-(Methylpiperazino)butyl)-2-chloroacridone |
| 9 | 10-(4′-N-Piperidinobutyl)-2-chloroacridone |
| 10 | 10-(4′-N-[(β-Hydroxyethyl)piperazino]butyl)-2- |
| | chloroacridone |
| 13 | 10-(4′-Chlorobutyl)-2-chloroacridone |
| 21 | 10-(4′-N-Pyrrolidinobutyl)-2-bromoacridone |
| 22 | 10-(4′-N-Morpholinobutyl)-2-bromoacridone |
| 25 | 10-(3′-N-Pyrrolidinopropyl)-2-bromoacridone |
| 26 | 10-(3′-[N-Diethylamino]propyl)-2-bromoacridone |

6.2. Synthesis of AKT Modulating Compounds

The phenoxazine compounds of formula (I) useful in the present invention can be generated synthetically by standard organic synthetic methods readily known to one of ordinary skill in the art. Suitable synthetic pathways are described in, for example, U.S. Pat. No. 5,371,081; Horton et al. Mol. Pharmacol. 1993;44:552-559; Eregowda et al. Asian J. Chem. 1999; 1:878-905; and Eregowda et al. Indian J. Chem. 2000;39B:243-259, the entire contents of each of which is hereby incorporated by reference in its entirety.

For example, the compounds of formula (I) may be prepared according to the following general synthetic scheme:

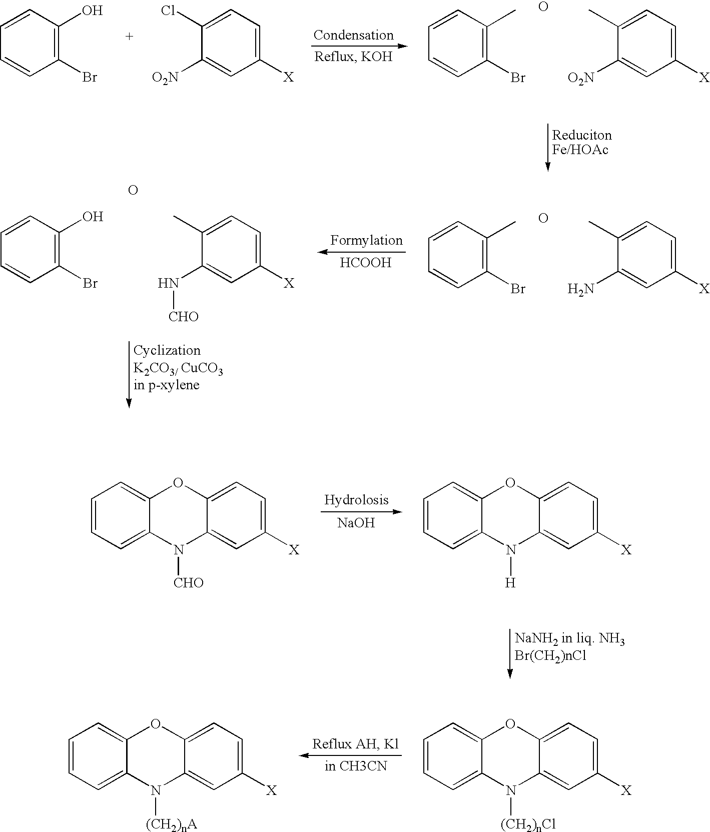

where A and X are as described herein.

In general, the synthesis of the compounds of formula (I) is straightforward. N-alkylation can be achieved in the presence of basic condensing agents like sodium amide. The general procedure for preparing the phenoxazine compounds of formula (I) consists of the condensation of the appropriately substituted phenoxazine with the appropriate α,ω-dialkylhalide, such as Cl(CH₂)ₙBr wherein n is 2 to 6, in the presence of sodium amide, either in liquid ammonia or in an anhydrous solvent such as toluene or benzene. For instance, the reaction of the phenoxazine with mixed chlorobromoalkanes in the presence of sodium amide gives reactive N-chloroalkylphenoxazines, which can then be converted to the desired compound by reaction with an intermediate of the formula H(CH₂)ₙA wherein n and A have the meanings set forth above.

The acridone compounds of formula (III) useful in the present invention can be generated synthetically by standard organic synthetic methods readily known to one of ordinary skill in the art. For example, synthetic pathways for acridones of formula (III) wherein K is alkoxy are described, for example, in Hegde et al. Eur. J. Med. Chem. 2004;39:161-177, while synthetic pathways for acridones of formula (III) wherein J is alkoxy are described, for example, in Krishnegowda et al. Biorg. Med. Chem. 2002; 10:2367-2380 (the contents of each of which is hereby incorporated by reference in its entirety). The novel acridones of formula (III) wherein J is halogen may be generated synthetically by standard organic synthetic methods readily known to one of ordinary skill in the art, for example as described in the Examples, Section 7.1 below.

For example, the compounds of formula (III) may be prepared according to the following general synthetic scheme:

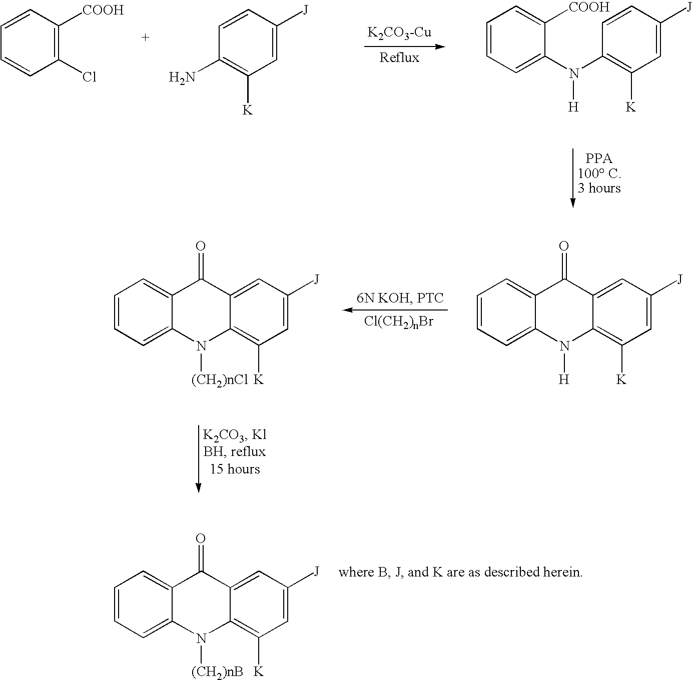

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Preferred pharmaceutically acceptable derivatives are salts, solvates, esters, carbamates and phosphate esters. Particularly preferred pharmaceutically acceptable derivatives are salts, solvates and esters. Most preferred pharmaceutically acceptable derivatives are salts and esters.

The term “salts” can include acid addition salts or addition salts of free bases. Preferably, the salts are pharmaceutically acceptable. Examples of acids which may be employed to form pharmaceutically acceptable acid addition salts include, but are not limited to, salts derived from nontoxic inorganic acids such as nitric, phosphoric, sulfuric, or hydrobromic, hydroiodic, hydrofluoric, phosphorous, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J Pharma. Sci. 1977;66:1).

A pharmaceutically acceptable salt of the phenoxazine and acridone compounds of the invention may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of Formula (I) and the resulting mixture evaporated to dryness (lyophilized) to obtain the acid addition salt as a solid. Alternatively, phenoxazine and acridone compounds may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form nontoxic salts and examples are hydrochloride, hydrobromide, hydroiodide, sulfate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulfonates (e.g. methanesulfonate, ethanesulfonate, benzenesulfonate or p-toluenesulfonate) and isethionate. Representative examples include trifluoroacetate and formate salts, for example the bis- or tris-trifluoroacetate salts and the mono or diformate salts, in particular the bis- or tris-trifluoroacetate salt and the monoformate salt.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the phenoxazine and acridone compounds are within the scope of the invention. The salts of the phenoxazine and acridone compounds may form solvates (e.g., hydrates) and the invention also includes all such solvates. The meaning of the word “solvates” is well known to those skilled in the art as a compound formed by interaction of a solvent and a solute (i.e., solvation). Techniques for the preparation of solvates are well established in the art (see, for example, Brittain. Polymorphism in Pharmaceutical solids. Marcel Decker, New York, 1999.).

The present invention also encompasses prodrugs of the phenoxazine and acridone compounds, i.e., compounds which release an active parent drug in vivo when administered to a mammalian subject. A prodrug is a pharmacologically active or more typically an inactive compound that is converted into a pharmacologically active agent by a metabolic transformation. Prodrugs of the phenoxazine and acridone compounds are prepared by modifying functional groups present in the compounds in such a way that the modifications may be cleaved in vivo to release the parent compound. In vivo, a prodrug readily undergoes chemical changes under physiological conditions (e.g., are acted on by naturally occurring enzyme(s)) resulting in liberation of the pharmacologically active agent. Prodrugs include phenoxazine and acridone compounds wherein a hydroxy, amino, or carboxy group of the compound is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of formula I or any other derivative which upon being brought to the physiological pH or through enzyme action is converted to the active parent drug. Conventional procedures for the selection and preparation of suitable prodrug derivatives are described in the art (see, for example, Bundgaard. Design of Prodrugs. Elsevier, 1985).

Prodrugs may be administered in the same manner as the active ingredient to which they convert or they may be delivered in a reservoir form, e.g., a transdermal patch or other reservoir which is adapted to permit (by provision of an enzyme or other appropriate reagent) conversion of a prodrug to the active ingredient slowly over time, and delivery of the active ingredient to the patient.

The present invention also encompasses metabolites. “Metabolite” of a phenoxazine or acridone compound disclosed herein is a derivative of a compound which is formed when the compound is metabolised. The term “active metabolite” refers to a biologically active derivative of a compound which is formed when the compound is metabolised. The term “metabolised” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyses a variety of oxidative and reductive reactions while uridine diphosphate glucuronyltransferases catalyse the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996), pages 11-17.

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

6.3. Uses of AKT Modulating Compounds

The AKT modulating phenoxazine and acridone compounds of the invention specifically and effectively modulate the kinase activity of AKT proteins and thereby modulate AKT-signal transduction in various types of cells. As discussed above, the AKT kinases are associated with a variety of physiological responses, including the inhibition of apoptosis and promotion of cell survival. Extensive evidence has demonstrated a crucial role for AKT in tumorigenesis, while activation of AKT has been shown to associate with tumor invasiveness and chemoresistance.

Accordingly, the invention further provides compositions for and methods of modulating AKT activity, inhibiting cell growth, treating cancer, treating transplant rejection, and treating coronary artery disease based upon the phenoxazine and acridone compounds of the invention.

Methods of Modulating AKT Activity

The invention provides compositions for and methods of modulating AKT activity using the phenoxazine and acridone compounds of the invention. By “modulating AKT activity” is meant any alteration in the function of AKT, including activating AKT activity and inhibiting AKT activity. As discussed above, preferred phenoxazine and acridone compounds of the invention have been shown to inhibit AKT activity. However, the invention also contemplates phenoxazine and acridone compounds that activate AKT activity.

By “AKT activity” is meant any function of AKT, including but not limited to AKT phosphorylation, AKT kinase activity, and AKT signaling to downstream targets such as mTOR, p70S6 kinase, and ribosomal protein S6 (rpS6 or S6).

AKT activity may be assessed by any of the methods well established in the art, including quantitation of AKT phosphorylation; quantitation of AKT kinase activity; determination of the cellular localization of AKT, quantitation of phosphorylation of AKT downstream targets such as mTOR, p70S6 kinase, S6 and GSK-3; and quantitation of the kinase activity of AKT downstream targets such as mTOR, p70S6 kinase, and GSK-3.

AKT phosphorylation may be quantitated, for example, using commercially available antibodies specific for phosphorylated residues of AKT. For example, antibodies specific for human and mouse AKT phosphorylated on residues Ser473, Thr308, Tyr326, or Ser505 are available from a variety of sources, including Biosource International, Covance Research Products, Abcam, Cell Signaling Technology, Novus Biologicals, and R&D Systems. Such antibodies may be used in any of the assays well established in the art, including immunoprecipitation, Western blotting, and ELISA. For example, ELISA kits for quantitation of AKT phosphorylated on residues Ser473 or Thr308 are available from a variety of sources, including Biosource International, Cell Signaling Technology, Sigma, and Calbiochem.

AKT kinase activity may be quantitated, for example, using an in vitro kinase assay. A variety of AKT kinase assay kits are commercially available, for example, from BioSource International, BioVision, Calbiochem, Cell Signaling Technology, Molecular Devices, Upstate Biotechnology, or Stressgen Biologicals. Peptide substrates of AKT for use in vitro AKT kinase activity assays are commercially available, for example, from BioSource International, Calbiochem, Cell Signaling Technology, and Upstate Biotechnology. AKT kinase assays may be performed as previously described (see, e.g., Nakatani et al. J Biol Chem 1999;274:21528-21532).

Cellular localization of AKT may be determined by any of the methods well known in the art, e.g. immunocytochemistry using any of the commercially available antibodies to AKT.

Protocols for the quantitation of phosphorylation and/or kinase activity of the AKT downstream targets mTOR, p70S6 kinase, S6 and GSK-3 are well established in the art. Phosphorylation of AKT downstream targets such as mTOR, p70S6 kinase, S6 and GSK-3 may be quantitated, for example, using commercially available antibodies. For example antibodies specific for phosphorylated residues of mTOR, p70S6 kinase, S6 or GSK-3 are available from a variety of sources, including Covance Research Products, Abcam, Cell Signaling Technology, Stressgen Bioreagents, Biosource International and Upstate Biotechnology. Such antibodies may be used in any of the assays well established in the art, including immunoprecipitation, Western blotting, and ELISA. ELISA kits for quantitation of phosphorylated GSK-3, for example, are available from Active Motif. ELISA kits for quantitation of phosphorylated p70S6 kinase, for example, are available from R&D Systems. Kinase activity of the AKT downstream targets mTOR, p70S6 kinase, and GSK-3 may be quantitated, for example, using an in vitro kinase assay. Such in vitro assays are well described in the art.

The method of modulating AKT activity comprises contacting an AKT with an effective amount of a phenoxazine or acridone compound of the invention. In one embodiment, the phenoxazine or acridone compound of the invention may be directly contacted to AKT, e.g., in vitro. In another embodiment, the phenoxazine or acridone compound of the invention may be contacted to a cell comprising AKT. Without intending to be limited by mechanism, it is thought that upon contact with the cell, the phenoxazine and acridone compounds of the invention are taken up by the cell, resulting in direct contact of the compound with AKT within the cell.

As used herein, a cell that comprises AKT is any cell that contains an AKT protein, including cells that endogenously express AKT and cells that ectopically express AKT. The target cells may be, for example, cells cultured in vitro or cells found in vivo in an organism, such as a mammal. In preferred embodiments, the cells are mammalian cells. In particularly preferred embodiments, the cells are cancer cells.

The AKT expression status of a cell may be determined by any of the techniques well established in the art including Western blotting, immunoprecipitation, flow cytometry/FACS, immunohistochemistry/immunocytochemistry, Northern blotting, RT-PCR, whole mount in situ hybridization, etc. For example, monoclonal and polyclonal antibodies to human and/or mouse AKT1, AKT2 or AKT3 are commercially available from a variety of sources, e.g., from BD Biosciences, Cell Signaling Technology, IMGENEX, Novus Biologicals, Calbiochem, and R&D Systems. Human and mouse AKT1, AKT2, or AKT3 primer pairs are commercially available, e.g., from Bioscience Corporation. SuperArray RT-PCR Profiling Kits for simultaneous quantitation of the expression of mouse or human AKT1, AKT2, and AKT3 are available from Bioscience Corporation.

By “effective amount” is meant an amount of a phenoxazine or acridone compound of the invention effective to modulate AKT activity. It is within the skill of one of ordinary skill in the art to identify such an effective amount, e.g., using the methods described above. In one embodiment, an effective amount is from about 1 μM to about 50 mM of a phenoxazine or acridone compound of the invention. In another embodiment, an effective amount is from about 1 μM to about 5 μM of a phenoxazine or acridone compound of the invention. In another embodiment, an effective amount is about 2.5 μM of a phenoxazine or acridone compound of the invention.

Methods of Inhibiting Cell Growth of a Cell.

The invention provides compositions for and methods of inhibiting cell growth using the phenoxazine and acridone compounds of the invention. As used herein the phrase “inhibiting cell growth” encompasses any effect that serves to inhibit an increase in cell number, including cytostatic effects (e.g., inhibition of cell division) and cytotoxic effects (e.g., promotion of apoptosis and promotion of necrosis). Methods for the evaluation of cell growth are well established in the art, including methods to quantitate cell number, methods to evaluate doubling time of a cell population, methods to evaluate progression of the cell division cycle (e.g., entry into S phase), and methods to identify and characterize cell death (e.g., trypan blue exclusion to assess cell viability). For example, kits for the quantitation of apoptosis are commercially available from a variety of sources including Upstate Biotechnology, Biovision, Sigma Aldrich, and Cambrex. Appropriate target cells for use in such a method include any cell that comprises an AKT protein (for a discussion of cells comprising AKT, see the section Methods of modulating AKT activity, above). The target cells may be, for example, cells cultured in vitro or cells found in vivo in an organism, such as a mammal.

The invention further provides compositions for and methods of inhibiting cell growth in a cell using the phenoxazine and acridone compounds of the invention, where the cell is a cell in which AKT is activated. Appropriate target cells for use in such a method include any cell in which AKT is activated. The target cells may be, for example, cells cultured in vitro or cells found in vivo in an organism, such as a mammal.

The term “a cell in which AKT is activated” refers to any cell in which AKT kinase activity is abnormally activated. AKT kinase activity may be abnormally activated, for example, as a result of duplication of an AKT gene, overexpression of an AKT gene or protein, or abnormal activation of an AKT signal transduction pathway. Such alterations in AKT activity may be detected in cells using any of the techniques well known in the art. See, for example, Staal Proc Natl Acad Sci USA 1987;84:5034-5037; Nakatani et al. J Biol Chem 1999;274:21528-21532; Ruggeri et al. Mol Carcinol 1998;21:81-86; Miwa et al. Biochem Biophys Res Com 1996;23:225-968-974; and Cheng et al. Proc Natl Acad Sci 1992;89:9267-9271.

For example, the level of AKT kinase activity in a cell may be quantitated, for example, using an in vitro kinase assay. A variety of AKT kinase assay kits are commercially available, for example, from BioSource International, BioVision, Calbiochem, Cell Signaling Technology, Molecular Devices, Upstate Biotechnology, or Stressgen Biologicals. Peptide substrates of AKT for use in vitro AKT kinase activity assays are commercially available, for example, from BioSource International, Calbiochem, Cell Signaling Technology, and Upstate Biotechnology. AKT kinase assays may be performed as previously described (see, e.g., Nakatani et al. J Biol Chem 1999;274:21528-21532).

In another example, the copy number of an AKT gene in a cell may be quantitated using standard techniques, including Southern blotting, quantitative PCR, fluorescence in situ hybridization of metaphase chromosome spreads, and other cytogenetic techniques. For example, AKT gene copy number may be estimated by Southern blot as previously described (see, e.g., Staal. Proc Natl Acad Sci USA 1987;84:5034-5037 and Cheng et al. Proc Natl Acad Sci USA 1992;89:9267-9271). A cell in which AKT is activated may show an increase in AKT gene copy number.

In another example, the level of AKT expression in a cell may be quantitated using any of the standard techniques well known in the art, including Western blotting, immunoprecipitation, flow cytometry/FACS, immunohistochemistry/immunocytochemistry, Northern blotting, RT-PCR, whole mount in situ hybridization, etc. For example, monoclonal and polyclonal antibodies to human and/or mouse AKT1, AKT2 or AKT3 are commercially available from a variety of sources, e.g., from BD Biosciences, Cell Signaling Technology, IMGENEX, Novus Biologicals, Calbiochem, and R&D Systems. Human and mouse AKT1, AKT2, or AKT3 primer pairs are commercially available, e.g., from Bioscience Corporation. SuperArray RT-PCR Profiling Kits for simultaneous quantitation of the expression of mouse or human AKT1, AKT2, and AKT3 are available from Bioscience Corporation. For example, AKT gene expression may be quantitated by Northern Blot, Western blot, or RT-PCR as previously described (see, e.g., Cheng et al. Proc Natl Acad Sci USA 1992;89:9267-9271; Nakatani et al. J Biol Chem 1999;273:21528-21532; and Massion et al. Am J Respi Crit Care Med 2004; 170:1088-1094). A cell in which AKT is activated may show an increase in AKT expression.

Abnormal activation of the AKT signal transduction pathway may result, for example, from an abnormal decrease in PTEN activity. Activation of AKT is negatively regulated by a tumor suppressor protein known as protein phosphatase and tensin homolog deleted on chromosome 10 (PTEN, also known as MMAC1 and TEP1), a tyrosine-threonine/lipid phosphatase that dephosphorylates the 3-position of PtdIns-3-phosphate. Amino acid and nucleotide sequences for PTEN have been reported for a variety of species, including human, mouse, rat, dog, chicken, Xenopus, zebrafish, and Drosophila. Exemplary nucleotide and amino acid sequences for human PTEN are set forth in SEQ ID NO: 9 and 10, respectively.

PTEN activity may be abnormally decreased, for example by mutation of the PTEN gene (e.g. by point mutation, deletion, and/or insertion), by reduced expression of the PTEN gene or protein (e.g. due to abnormal promoter methylation), or by abnormal inhibition of the phosphatase activity of PTEN. Protocols for the detection of alterations in PTEN are well established in the art, including methods to detect PTEN gene deletions and mutations (see, e.g., Whang et al. Proc Natl Acad Sci USA 1998;95:5246-5250; Steck et al. Nat Genet 1997; 15:356-362; Liaw et al. Nature Genet 1997; 16:64-67; and Li et al. Science 1997;275:1943-1947), methods to detect a reduction in expression of PTEN mRNA or protein (see, e.g., Whang et al. Proc Natl Acad Sci USA 1998;95:5246-5250 and Altomare et al. J Cell Biochem 2003;88:470-476), and methods to detect PTEN gene silencing due to alterations in promoter methylation (see, e.g., Kang et al. Lab Invest 2002;82:285-291 and Sato et al. Virchows Arch. 2002;440: 160-5). Kits for the quantitation of PTEN phosphatase activity are commercially available, for example, from Upstate Biotechnology and Echelon Biosciences. Kits for the quantitation of human, rat, or mouse PTEN protein levels by ELISA are commercially available, for example, from R&D Systems.

Cells in which PTEN activity is abnormally decreased include glioblastomas, endometrial cancer, breast cancer, thyroid cancer, prostate cancer, cervical cancer, colorectal cancer, lung cancer, and gastric cancer. PTEN activity is abnormally decreased in the human hereditary cancer predisposition syndromes Cowden Disease and Bannayan-Zonana syndrome.

Abnormal activation of the AKT signal transduction pathway may result, for example, from an abnormal increase in PI 3-kinase activity. Activation of AKT is positively regulated by phosphatidylinositol 3-kinase (PI 3-kinase). PI 3-kinase itself phosphorylates PtdIns to generate PtdIns-3-phosphates. PI 3-kinase-generated phospholipids activate AKT by multiple mechanisms, including direct binding of phosphoinositides to the pleckstrin homology domain of AKT and translocation of AKT from the cytoplasm to the nucleus.

Surface receptor-activated PI 3-kinases function in mammals (e.g. mice), insects (e.g. Drosophila melanogaster), nematodes (e.g. Caenorhabditis elegans) and slime mold, but not yeast.

PI 3-kinase is a heterodimeric enzyme, consisting of a catalytic and a regulatory subunit. At least five isoforms of the regulatory subunit have been identified and classified into three groups comprising 85-kDa (Class I), 55-kDa (Class II), and 50-kDa (Class III) proteins. At least four isoforms of the catalytic subunit have been identified: p110α, p110β, p110γ, and p110δ, and there is a growing literature describing distinct biological functions for these proteins. Thus, Class I PI 3-kinase is composed of a regulatory p85 subunit (e.g. p85α or p85β), and a catalytic p110 (e.g. p110α, p110β, p110γ, or p110δ) subunit. In preferred embodiments, the PI 3-kinase is a mammalian PI 3-kinase. In preferred embodiments, the PI 3-kinase is a Class I PI 3-kinase. In particularly preferred embodiments, the PI 3-kinase is a mammalian Class I PI 3-kinase.

For example, the genes encoding p85 regulatory subunits and p110 catalytic subunits have been identified in a variety of species, including human, mouse, rat, and zebrafish. For example, human p85α is encoded by the PIK3R1 gene (see, e.g., GenBank Accession numbers NM_{—}181504, NM_{—}181523, and NM_{—}181524); human p85β is encoded by the PIK3R2 gene (see, e.g., GenBank Accession numbers X80907 and NM_{—}005207); human p110α is encoded by the PIK3CA gene (see, e.g., GenBank Accession numbers NM_{—}006218 and U79143); human p110β is encoded by the PIK3CB gene (see, e.g., GenBank Accession numbers NM_{—}006219 and S67334); human p110γ is encoded by the PIK3CG gene (see, e.g., GenBank Accession number NM_{—}002649), human p110δ is encoded by the PIK3CD gene (see, e.g., GenBank Accession numbers NM_{—}005026 and U86453).

PI 3-kinase activity may be abnormally increased, for example by gene duplication of a PIK3R or a PIK3C gene, by increased expression of a PIK3R or a PIK3C gene or protein, or by abnormal activation of the kinase activity of PI 3-kinase.

In vitro assays for PI 3-kinase activity may be performed, for example, as previously described (see, e.g., Moore et al. Cancer Res 1998;58:5239-5247; Shayesteh et al. Nat. Genet. 1999;21:99-102; and Altomare et al. J Cell Biochem 2003;88:470-476). Kits for quantitation of PI 3-kinase protein are commercially available, including ELISA-based kits (e.g., from AG Scientific or Echelon Biosciences) and fluorescence polarization-based kits (e.g., Echelon Biosciences).

Gene duplications of PIK3R or PIK3C genes, for example, may be detected as previously described (see, e.g., Byun et al. Int J Cancer 2003; 104:318-327; Shayesteh et al. Nat. Genet. 1999;21:99-102; Ma et al. Oncogene 2000; 19:2739-2744; Knobbe and Reifenberger. Brain Pathol 2003; 13:507-518; Massion et al. Am J Respi Crit Care Med 2004;170:1088-1094; and Gao et al. Am J Physiol Cell Physiol 2004;287:C281-291).

Increased expression of PIK3R or PIK3C genes, for example may be detected as previously described (see, e.g., Shayesteh et al. Nat. Genet. 1999;21:99-102; Gershtein et al. Clin Chim Acta 1999;287:59-67; Salh et al. Int J Cancer 2002, 98:148-154; and Knobbe and Reifenberger. Brain Pathol 2003;13:507-518). Antibodies specific for the various regulatory and catalytic subunits of PI 3-kinase are commercially available from a variety of sources, including AG Scientific, Biomeda, Upstate Biotechnology, and Cell Signaling Technology.

The method of inhibiting cell growth of a cell comprises contacting the cell with an effective amount of a phenoxazine or acridone compound of the invention. In preferred embodiments, the cells are mammalian cells. In particularly preferred embodiments, the cells are cancer cells.

The method of inhibiting cell growth of a cell, wherein the cell is a cell in which AKT is activated, comprises contacting the cell with an effective amount of a phenoxazine or acridone compound of the invention. In preferred embodiments, the cells are mammalian cells. In particularly preferred embodiments, the cells are cancer cells.

By “effective amount” is meant an amount of a phenoxazine or acridone compound of the invention effective to inhibit cell growth. It is within the skill of one of ordinary skill in the art to identify such an effective amount, e.g., using the methods described above. In one embodiment, an effective amount is from 100 nM to 50 mM of a phenoxazine or acridone compound of the invention. In another embodiment, an effective amount is from 100 nm to 25 μM of a phenoxazine or acridone compound of the invention. In another embodiment, an effective amount is from 2 μM to 6 μM of a phenoxazine or acridone compound of the invention.

Methods of Treating Cancer.

The invention also provides compositions for and methods of treating cancer in a patient using the phenoxazine and acridone compounds of the invention.

By “treating cancer” is meant any amelioration of the clinical symptoms of cancer, including but not limited to, tumor size, number of tumors, tumor invasiveness, tumor metastasis, tumor angiogenesis, and/or tumor recurrence. Thus the methods of the invention encompass uses of the phenoxazine or acridone compounds of the invention to prevent cancer (e.g. to prevent neoplasm, to prevent progression to malignancy, etc.), to treat an existing cancer (e.g., to reduce tumor size or number), and to prevent recurrence of a cancer (e.g., following surgery, radiation therapy, chemotherapy, bone marrow transplant, or other intervention to treat a cancer). In the methods of the invention, the phenoxazine or acridone compounds of the invention may be administered in conjunction with other cancer therapies, such as surgery, chemotherapy, radiation therapy, bone marrow transplant, etc. In such combination therapies the phenoxazine or acridone compounds may be administered prior to, concurrent with, or subsequent to the other cancer therapy.

The invention further provides compositions for and methods of treating cancer in a patient using the AKT inhibiting phenoxazine and acridone compounds of the invention, where the cancer is a cancer in which AKT is activated.

The term “a cancer in which AKT is activated” refers to any cancer in which AKT kinase activity is abnormally activated. AKT kinase activity may be abnormally activated, for example, as a result of duplication of an AKT gene, overexpression of an AKT gene or protein, or abnormal activation of an AKT signal transduction pathway. Such alterations in AKT activity may be detected in cancer cells using any of the techniques well known in the art. See, for example, Staal Proc Natl Acad Sci USA 1987;84:5034-5037; Nakatani et al. J Biol Chem 1999;274:21528-21532; Ruggeri et al. Mol Carcinol 1998;21:81-86; Miwa et al. Biochem Biophys Res Com 1996;23:225-968-974; and Cheng et al. Proc Natl Acad Sci 1992;89:9267-9271.

For example, the level of AKT kinase activity in a cancer cell may be quantitated, for example, using an in vitro kinase assay. A variety of AKT kinase assay kits are commercially available, for example, from BioSource International, BioVision, Calbiochem, Cell Signaling Technology, Molecular Devices, Upstate Biotechnology, or Stressgen Biologicals. Peptide substrates of AKT for use in vitro AKT kinase activity assays are commercially available, for example, from BioSource International, Calbiochem, Cell Signaling Technology, and Upstate Biotechnology. AKT kinase assays may be performed as previously described (see, e.g., Nakatani et al. J Biol Chem 1999;274:21528-21532).

In another example, the copy number of an AKT gene in a cancer cell may be quantitated using standard techniques, including Southern blotting, quantitative PCR, fluorescence in situ hybridization of metaphase chromosome spreads, and other cytogenetic techniques. For example, AKT gene copy number may be estimated by Southern blot as previously described (see, e.g., Staal. Proc Natl Acad Sci USA 1987;84:5034-5037 and Cheng et al. Proc Natl Acad Sci USA 1992;89:9267-9271). A cancer in which AKT is activated may show an increase in AKT gene copy number.

In another example, the level of AKT expression in a cancer may be quantitated using any of the standard techniques well known in the art, including Western blotting, immunoprecipitation, flow cytometry/FACS, immunohistochemistry/immunocytochemistry, Northern blotting, RT-PCR, whole mount in situ hybridization, etc. For example, monoclonal and polyclonal antibodies to human and/or mouse AKT1, AKT2 or AKT3 are commercially available from a variety of sources, e.g., from BD Biosciences, Cell Signaling Technology, IMGENEX, Novus Biologicals, Calbiochem, and R&D Systems. Human and mouse AKT1, AKT2, or AKT3 primer pairs are commercially available, e.g., from Bioscience Corporation. SuperArray RT-PCR Profiling Kits for simultaneous quantitation of the expression of mouse or human AKT1, AKT2, and AKT3 are available from Bioscience Corporation. For example, AKT gene expression may be quantitated by Northern Blot, Western blot, or RT-PCR as previously described (see, e.g., Cheng et al. Proc Natl Acad Sci USA 1992;89:9267-9271; Nakatani et al. J Biol Chem 1999;273:21528-21532; and Massion et al. Am J Respi Crit Care Med 2004; 170:1088-1094). A cancer in which AKT is activated may show an increase in AKT expression.

Cancers in which AKT has been shown to be abnormally activated include gastric adenocarcinoma, breast cancer, ovarian cancer, pancreatic cancer, prostate cancer, and chronic myelogenous leukemia.

Abnormal activation of the AKT signal transduction pathway may result, for example, from an abnormal decrease in PTEN activity. For a discussion of PTEN, see the section Methods of inhibiting cell growth, above.

PTEN activity may be abnormally decreased, for example by mutation of the PTEN gene (e.g. by point mutation, deletion, and/or insertion), by reduced expression of the PTEN gene or protein (e.g. due to abnormal promoter methylation), or by abnormal inhibition of the phosphatase activity of PTEN. Protocols for the detection of alterations in PTEN are well established in the art, including methods to detect PTEN gene deletions and mutations (see, e.g., Whang et al. Proc Natl Acad Sci USA 1998;95:5246-5250; Steck et al. Nat Genet 1997; 15:356-362; Liaw et al. Nature Genet 1997; 16:64-67; and Li et al. Science 1997;275:1943-1947), methods to detect a reduction in expression of PTEN mRNA or protein (see, e.g., Whang et al. Proc Natl Acad Sci USA 1998;95:5246-5250 and Altomare et al. J Cell Biochem 2003;88:470-476), and methods to detect PTEN gene silencing due to alterations in promoter methylation (see, e.g., Kang et al. Lab Invest 2002;82:285-291 and Sato et al. Virchows Arch. 2002;440: 160-5). Kits for the quantitation of PTEN phosphatase activity are commercially available, for example, from Upstate Biotechnology and Echelon Biosciences. Kits for the quantitation of human, rat, or mouse PTEN protein levels by ELISA are commercially available, for example, from R&D Systems.

Cancers in which PTEN activity is abnormally decreased include glioblastomas, endometrial cancer, breast cancer, thyroid cancer, prostate cancer, cervical cancer, colorectal cancer, lung cancer, and gastric cancer. PTEN activity is abnormally decreased in the human hereditary cancer predisposition syndromes Cowden Disease and Bannayan-Zonana syndrome.

Abnormal activation of the AKT signal transduction pathway may result, for example, from an abnormal increase in PI 3-kinase activity. For a discussion of PI 3-kinase, see the section Methods of inhibiting cell growth, above.

PI 3-kinase activity may be abnormally increased, for example by gene duplication of a PIK3R or a PIK3C gene, by increased expression of a PIK3R or a PIK3C gene or protein, or by abnormal activation of the kinase activity of PI 3-kinase.

In vitro assays for PI 3-kinase activity may be performed, for example, as previously described (see, e.g., Moore et al. Cancer Res 1998;58:5239-5247; Shayesteh et al. Nat. Genet. 1999;21:99-102; and Altomare et al. J Cell Biochem 2003;88:470-476). Kits for quantitation of PI 3-kinase protein are commercially available, including ELISA-based kits (e.g. from AG Scientific or Echelon Biosciences) and fluorescence polarization-based kits (e.g., Echelon Biosciences).

Gene duplications of PIK3R or PIK3C genes, for example, may be detected as previously described (see, e.g., Byun et al. Int J Cancer 2003; 104:318-327; Shayesteh et al. Nat. Genet. 1999;21:99-102; Ma et al. Oncogene 2000; 19:2739-2744; Knobbe and Reifenberger. Brain Pathol 2003;13:507-518; Massion et al. Am J Respi Crit Care Med 2004;170:1088-1094; and Gao et al. Am J Physiol Cell Physiol 2004;287:C281-291).

Increased expression of PIK3R or PIK3C genes, for example may be detected as previously described (see, e.g., Shayesteh et al. Nat. Genet. 1999;21:99-102; Gershtein et al. Clin Chim Acta 1999;287:59-67; Salh et al. Int J Cancer 2002, 98:148-154; and Knobbe and Reifenberger. Brain Pathol 2003; 13:507-518). Antibodies specific for the various regulatory and catalytic subunits of PI 3-kinase are commercially available from a variety of sources, including AG Scientific, Biomeda, Upstate Biotechnology, and Cell Signaling Technology.

Cancers in which PI 3-kinase activity is abnormally increased include ovarian cancer, breast cancer, epithelial carcinoma of the mouth, lung cancer, gastric carcinoma, cervical cancer, and glioblastoma.

Appropriate patients to be treated according to the methods of the invention include any animal in need of such treatment. Methods for the diagnosis and clinical evaluation of cancer are well established in the art. Thus, it is within the skill of the ordinary practitioner in the art (e.g., a medical doctor or veterinarian) to determine if a patient is in need of treatment for cancer.

The method of treating cancer in a patient comprises administering to a patient in need of such treatment an effective amount of a phenoxazine or acridone compound of the invention. In preferred embodiments, the patient is a mammal. In particularly preferred embodiments, the patient is a human.

The method of treating cancer in a patient, wherein the cancer is a cancer in which AKT is activated, comprises administering to a patient in need of such treatment an effective amount of a phenoxazine or acridone compound of the invention. In preferred embodiments, the patient is a mammal. In particularly preferred embodiments, the patient is a human.

By “effective amount” is meant an amount of a phenoxazine or acridone compound of the invention sufficient to result in a therapeutic response. The therapeutic response can be any response that a user (e.g., a clinician) will recognize as an effective response to the therapy. The therapeutic response will generally be an amelioration of one or more symptoms of a cancer, e.g., a reduction in the number of cancer cells observed, e.g., in a biopsy from a patient during treatment or a reduction in tumor size and/or number. Data obtained from cell culture assay or animal studies may be used to formulate a range of dosages for use in humans. It is further within the skill of one of ordinary skill in the art to determine an appropriate treatment duration, and any potential combination treatments, based upon an evaluation of therapeutic response. For example, a phenoxazine or acridone compound of the invention may be used in any of the therapeutic regimens well known in the art for chemotherapeutic drugs.

AKT Inhibitory Compounds and Immunosuppression

Rejection of transplanted tissue is a common clinical problem following transplant surgery. This rejection results from recognition of the transplanted tissue as “non-self” by the recipient's immune system, and subsequent mounting of an immune response, including cytotoxic T-cell responses, against the transplanted tissue. Therefore, transplant surgery patients are commonly placed on regimens of immunosuppressive drugs following transplant surgery. The commonly used immunosuppressive drugs include a class of agents known as calcineurin inhibitors (CNIs). Although CNIs, such as cyclosporin A, are potent inhibitors of T-cell proliferation, their interference with calcium functioning and mobilization has been associated with damage to the transplanted tissue (Easton and Houghton. Exp Op Ther Tar 2004:8:551-564). There is also some evidence that CNIs may be involved in the development of post-transplant diabetes (Davidson et al. Transplantation 2003:75:SS3-SS24).

As an alternative to classical CNIs, mTOR inhibitors, such as rapamycin and its analogs, are being developed for use as immunosuppressive agents following transplant surgery, including cardiac transplant (see, e.g., Keogh et al. Circulation 2004; 110:2694-2700) and renal transplant (see, e.g., Casas-Melley et al. Pediatr Transplant 2004;8:362-366). Inhibitors of mTOR block T-cell proliferation in response to IL-2, but have no effect on other steps leading to T-cell activation (Kuo et al. Nature 1992;358:70-73). Other studies suggest that mTOR inhibitors effect both the proliferation of dendritic cells and the ability of certain dendritic cells to present antigen (Hackstein et al. Blood 2003;101:4457-4463 and Chiang et al. J Immunol 2004;172:1355). Thus, mTOR inhibitors represent a class of immunosuppressive agents with a desirable clinical profile, i.e., suppression of an immune response against the transplanted tissue without undesirable side effects on transplant tissue viability.

As discussed herein, mTOR is a downstream target of AKT signaling, such that inhibition of AKT activity results in inhibition of mTOR activity. As discussed below, the AKT inhibiting phenoxazine and acridone compounds of the invention inhibit phosphorylation of mTOR. Thus, the invention provides compositions for and methods of inhibiting mTOR activity using the phenoxazine and acridone compounds of the invention. The novel phenoxazine and acridone compounds of the invention will also find utility in therapeutic regimens as immunosuppressive agents following transplant surgery.

Accordingly, the invention provides compositions for and methods of treating transplant rejection in a patient using the phenoxazine and acridone compounds of the invention. By “treating transplant rejection” is meant any amelioration of the clinical symptoms of transplant rejection, including but not limited to, mounting of an immune response to the transplanted tissue (e.g., B-cell or T-cell mediated responses such as antibody or cytotoxic T-cell responses) and damage to the transplanted tissue (e.g., tissue necrosis or lack of tissue function such as renal failure in the case of kidney transplant or heart failure in the case of heart transplant).

Stimulation of an immune response in a patient can be measured by standard tests including, but not limited to, the following: detection of transplanted tissue-specific antibody responses, detection of transplanted tissue-specific T-cell responses, including cytotoxic T-cell responses, direct measurement of peripheral blood lymphocytes; natural killer cell cytotoxicity assays (Provinciali et al. J. Immunol. Meth. 1992;155:19-24), cell proliferation assays (Vollenweider et al. J. Immunol. Meth. 1992;149:133-135), immunoassays of immune cells and subsets (Loeffler et al. Cytom. 1992; 13:169-174; and Rivoltini et al. Can. Immunol. Immunother. 1992;34:241-251); and skin tests for cell mediated immunity (Chang et al. Cancer Res. 1993;53:1043-1050). For an excellent text on methods and analyses for measuring the strength of the immune system, see, for example, Coligan et al., eds. Current Protocols in Immunology, Vol. 1 (Wiley & Sons: 2000).

Damage to the transplanted tissue may be characterized, for example, by direct examination of the transplanted tissue itself (e.g., and the cellular or molecular level) and/or by clinical evaluation of the transplant recipient. Protocols and methods for the clinical evaluation of transplant recipients and function of transplanted tissue following transplant surgery are well established in the art.

Suitable patients for the methods of the invention include any animal comprising a transplanted tissue, including heart, liver, kidney, lung, hematopoeitic cell, pancreatic beta islet cell, and basal ganglia cell transplant recipients. In the methods of the invention, the phenoxazine or acridone compounds of the invention may be administered in conjunction with other immunosuppressive therapies, e.g., in conjunction with CNI drug therapy. In such combination therapies the phenoxazine or acridone compounds may be administered prior to, concurrent with, or subsequent to the other immunosuppressive therapy.

The method of treating transplant rejection in a patient comprises administering to a patient in need of such treatment an effective amount of a phenoxazine or acridone compound of the invention. In preferred embodiments, the patient is a mammal. In particularly preferred embodiments, the patient is a human.

By “effective amount” is meant an amount of a phenoxazine or acridone compound of the invention sufficient to result in a therapeutic response. The therapeutic response can be any response that a user (e.g., a clinician) will recognize as an effective response to the therapy. The therapeutic response will generally be an amelioration of one or more symptoms of transplant rejection, e.g., reduction of a immune response to the transplanted tissue or improved function of the transplanted tissue. Data obtained from cell culture assay or animal studies may be used to formulate a range of dosages for use in humans. It is further within the skill of one of ordinary skill in the art to determine an appropriate treatment duration, and any potential combination treatments, based upon an evaluation of therapeutic response. For example, a phenoxazine or acridone compound of the invention may be used in any of the therapeutic regimens well known in the art for other immunosuppressive drugs, such as CNIs or rapamycin.

For example, the phenoxazine and acridone compounds of the invention may be used for prevention of acute renal allograft rejection. Protocols for diagnosis of, and immunosuppressive therapy for, acute renal allograph rejection are well known in the art (see, e.g., Hong and Kahan. Transplantation. 2001;71:1579-84). In such a regimen, in order to reverse ongoing rejection, the phenoxazine or acridone compounds of the invention may be administered to renal transplant recipients showing failure of conventional immunosuppressive regimens including, e.g., full courses of antilymphocyte sera. Such renal transplantation recipients may display either Grade IIB or Grade III biopsy-proven (Banff 1993 criteria) ongoing rejection episodes despite prior treatment, e.g. with pulse and/or oral recycling of steroids and/or a least one 14- to 21-day course of murine (OKT3) or equine (ATGAM) antilymphocyte treatment. In such a regimen, the efficacy of the phenoxazine and acridone compounds of the invention is preferably comparable to that of a known immunosuppressive therapy regimen. For example, the actual 12-month outcomes of two demographically similar cohorts of patients treated for refractory rejection with either a phenoxazine or acridone compound of the invention (Group I) or mycophenolate mofetil (MMF) added to a baseline regimen of cyclosporine (CsA)/prednisone (Pred) (Group II, representing treatment in a well characterized immunosuppressive regimen) may be compared. Successful rescue therapy will reverse the renal dysfunction in patients in Group I to a comparable extent as Group II. As a measure of renal function, mean serum creatinine values may be compared between groups. Successful immunosuppressive therapy will yield comparable 1-year patient and graft survival rates between Group I and Group II.

AKT Inhibitory Compounds and Coronary Artery Disease

The development of balloon angioplasty and later the use of metal stents to maintain luminal volume revolutionized the treatment of coronary artery disease. The major remaining obstacle to achieving long term success rates of greater than 80% for balloon angioplasty is restinosis (narrowing) of the artery as a result of migration and proliferation of vascular smooth muscle cells (for a review, see Easton and Houghton. Exp Op Ther Tar 2004:8:551-564).

In vitro and in vivo studies have shown that mTOR is a regulator of cell growth and proliferation of smooth muscle cells (for a review, see Easton and Houghton. Exp Op Ther Tar 2004:8:551-564). As a result of these studies, drug eluting stents containing the mTOR inhibitor rapamycin have been developed and evaluated in clinical trials (see, e.g., Morice et al. N Engl J Med 2002;346:1773-1780). Rapamycin stents are dramatically successful in preventing restinosis, such that such stents have become the standard of care for angioplasty patients.

As discussed herein, mTOR is a downstream target of AKT signaling, such that inhibition of AKT activity results in inhibition of mTOR activity. As discussed below, the AKT inhibiting phenoxazine and acridone compounds of the invention inhibit phosphorylation of mTOR. Like other mTOR inhibitors, the novel phenoxazine and acridone compounds of the invention will also find utility in drug eluting stents used for the treatment of coronary artery disease, such as restinosis following angioplasty.

Accordingly, the invention provides a drug eluting stent comprising a phenoxazine or acridone compound of the invention. The drug eluting stents of the invention may be formulated by techniques well established in the art (see, e.g., Morice et al. N Engl J Med 2002;346:1773-17; Tanabe et al. Circulation 2003;107:559-564; Kastrati et al. JAMA 2005;293:165-171; Yang and Moussa CAMJ2005;172:323-325; Perin Rev Cardiovasc Med 2005;6 SUPPL 1:S13-S21; and Williams and Kereiakes Rev Cardiovasc Med 2005;6 SUPPL 1:S22-S30). Coronary stents which may be loaded with the phenoxazine and acridone compounds of the invention are commercially available, e.g., from Guidant, Cordis, Boston Scientific, and Medtronic.

For example, a TAXUS NIRx-eluting stent (Boston Scientific Corporation) may be infused with a phenoxazine or acridone compound incorporated into a slow-release copolymer carrier system that gives biphasic release. For example, the total load of phenoxazine or acridone compound may be 1.0 μg/mm 2. For such stents, the initial release is over the first 48 hours followed by slow release over the next 10 days. For example, such stents may be 15 mm long and 3.0 or 3.5 mm in diameter.

In another example, a phenoxazine or acridone compound may be blended in a mixture of nonerodable polymers, and a layer of phenoxazine or acridone-polymer matrix with a thickness of 5 μM applied to the surface of a stainless-steel, balloon expandable stent (Bx Velocity, Cordis, Johnson & Johnson). The stent may be loaded with a fixed amount of phenoxazine or acridone compound per unit of metal surface area (e.g., 140 μg of phenoxazine or acridone per square centimeter). A layer of drug-free polymer may be applied on top of the drug-polymer matrix as a diffusion barrier to prolong release of the drug. The stent may, for example, release approximately 80 percent of the drug within 30 days of implantation.

The invention further provides compositions for and methods of treating coronary artery disease in a patient by placing a drug-eluting stent of the invention in a coronary artery of the patient. By “treating coronary artery disease” is meant any amelioration of the clinical symptoms of coronary artery disease including but not limited to migration and/or proliferation of vascular smooth muscle cells within a coronary artery, narrowing or occlusion of a coronary artery, inflammation of a coronary artery, and acute myocardial infarction.

Suitable patients for the methods of the invention include any animal in need of treatment for coronary artery disease, including any animal in need of balloon angioplasty. Protocols and methods for the diagnosis and evaluation of coronary artery disease are well established in the art.

The method of treating coronary artery disease in a patient comprises administering to a patient in need of such treatment a drug-eluting stent comprising an effective amount of a phenoxazine or acridone compound of the invention, wherein the administering comprises placing the drug-eluting stent within the luminal space of at least one coronary artery of the patient. In preferred embodiments, the patient is a mammal. In particularly preferred embodiments the patient is a human

By “effective amount” is meant an amount of a phenoxazine or acridone compound of the invention sufficient to result in a therapeutic response. The therapeutic response can be any response that a user (e.g., a clinician) will recognize as an effective response to the therapy. The therapeutic response will generally be an amelioration of one or more symptoms of coronary artery disease, e.g., attenuation or prevention of coronary artery narrowing. Data obtained from cell culture assay or animal studies may be used to formulate a range of dosages for use in humans. It is further within the skill of one of ordinary skill in the art to determine an appropriate treatment duration, and any potential combination treatments, based upon an evaluation of therapeutic response.

For example, a phenoxazine or acridone compound of the invention may be used in any of the regimens well known in the art for treatment of coronary artery disease using stents, especially following balloon angioplasty. For example, drug-infused stents of the invention may be administered to patients with coronary artery disease, and in particular to patients undergoing angioplasty, according to techniques well established in the art (see, e.g., Morice et al. N Engl J Med 2002;346:1773-17; Tanabe et al. Circulation 2003;107:559-564; Kastrati et al. JAMA 2005;293:165-171; Yang and Moussa CMAJ 2005;172:323-325; Perin Rev Cardiovasc Med 2005;6 SUPPL 1S13-S21; and Williams and Kereiakes Rev Cardiovasc Med 2005;6 SUPPL 1:S22-S30). For example, balloon predilation may be performed on a patient suffering from coronary artery disease. Thereafter, a NIRx-eluting stent with a load of a phenoxazine or acridone compound may implanted in the artery using conventional techniques. Postdilation may be performed if necessary. Periprocedural intravenous heparin may be given to maintain an activated clotting time ≧250 seconds, and patients may receive aspirin (e.g., at least 75 mg) and clopidogrel (e.g., 300 mg loading dose followed by 75 mg once daily for 6 months).

6.4. Therapeutic Compositions and Regimens

While it is possible that, for use in the methods of the invention, the phenoxazine and acridone compounds may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice.

Compositions used in this invention can be administered (e.g., in vitro or ex vivo to cell cultures, or in vivo to an organism) at therapeutically effective doses as part of a therapeutic regimen, e.g., for treating cancer or other disorders associated with AKT signaling. Accordingly, the invention also provides pharmaceutical preparations for use in the treatment of such disorders.

The terms “therapeutically effective dose” and “effective amount” refer to the amount of the compound that is sufficient to result in a therapeutic response. The therapeutic response can be any response that a user (e.g., a clinician) will recognize as an effective response to the therapy. Thus, the therapeutic response will generally be an amelioration of one or more symptoms of a disease or disorder.

Toxicity and therapeutic efficacy of compounds can be determined by standard pharmaceutical procedures, for example in cell culture assays or using experiments animals to determine the LD₅₀ and the ED₅₀. The parameters LD₅₀ and ED₅₀ are well known in the art, and refer to the doses of a compound that are lethal to 50% of a population, and therapeutically effective in 50% of a population, respectively. The dose ratio between toxic and therapeutic effects is referred to as the therapeutic index, and can be expressed as the ratio LD₅₀/ED₅₀. Compounds that exhibit large therapeutic indices are preferred. Nevertheless, compounds that exhibit toxic side effects may also be used. In such instances, however, it is particularly preferable to use delivery systems that specifically target such compounds to the site of affected tissue so as to minimize potential damage to other cells, tissues, or organs, and to reduce side effects.

Data obtained from cell culture assay or animal studies may be used to formulate a range of dosages for use in humans. The dosage of compounds used in therapeutic methods of the invention preferable lies within a range of circulating concentrations that includes the ED₅₀ concentration, but with little or no toxicity (i.e., below the LD₅₀ concentration). The particular dosage used in any application may vary within this range, depending upon factors such as the particular dosage form employed, the route of administration utilized, the conditions of the individual (e.g., the patient) and so forth.

A therapeutically effective dose may be initially estimated from cell culture assays and formulated in animal models to achieve circulating concentration ranges that include the IC₅₀. The IC₅₀ concentration of a compound is the concentration that achieves a half-maximal inhibition of symptoms (e.g., as determined from the cell culture assays). Appropriate dosages for use in a particular individual, for example in human patients, may then be more accurately determined using such information. Measures of compounds in plasma may be routinely measured in an individual such as a patient by techniques such as high performance liquid chromatography (HPLC) or gas chromatography.

Pharmaceutical compositions for use in this invention may be formulated in a conventional manner using one or more physiologically acceptable carriers or excipients. The phrase “pharmaceutically acceptable” refers to molecular entities and compositions that are generally regarded as safe. In particular, pharmaceutically acceptable carriers and excipients used in the pharmaceutical compositions of this invention are physiologically tolerable and do not typically produce an allergic or similar untoward reaction (for example, gastric upset, dizziness and the like) when administered to a patient or other individual. Preferred pharmaceutically acceptable carriers and excipients are approved by a government regulatory agency, such as the United States Food and Drug Administration (the “FDA”) and/or listed in the U.S. Pharmacopeia or other generally recognized Pharmacopeia for use in animals and, more preferably, in humans.

The term “carrier” refers to substances such as a diluent, adjuvant, excipient or other vehicle with which a compound of the invention is administered. Exemplary pharmaceutical carriers include, but are not limit to, sterile liquids such as water and oils, including those of petroleum, animal, vegetable or synthetic origin; for example, peanut oil, soybean oil, mineral oil, sesame oil and the like. Water or aqueous solutions, such as aqueous saline, dextrose and/or glycerol solutions, are preferably employed as carriers, particularly for injectable solutions. Alternatively, the carrier can be a solid dosage form carrier, including but not limited to one or more of a binder (e.g., for compressed pills), a glidant, an encapsulating agent, a flavorant, and/or a colorant. Other suitable pharmaceutical carriers are described, e.g., in Martin, E. W., Remington's Pharmaceutical Sciences, 20th Edition (Mack Publishing Company, Easton Pa., 2000).

The compounds of this invention, or their pharmaceutically acceptable salts and solvates, may be formulated for administration, e.g., by inhalation or insufflation (either through the mouth or the nose), or for oral, buccal, parenteral or rectal administration.

There may be different composition/formulation requirements depending on the different delivery systems. It is to be understood that not all of the compounds need to be administered by the same route. Likewise, if the composition comprises more than one active component, then those components may be administered by different routes. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions; or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For example, where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile. For example, the the phenoxazine and acridone compounds may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

Compounds of the invention can also be formulated as a depot preparation. Such long acting formulations may be administered by implantation (for example subcutaneous or intramuscular implantation) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that may contain one or more unit dosage forms containing the active ingredient. The pack can, for example, comprise metal or plastic foil, such as a blister pack. The pack or dispenser device can be accompanied by instructions for administration.

### 7. EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing the invention in spirit or in scope. The invention is therefore to be limited only by the terms of the appended claims along with the full scope of equivalents to which those claims are entitled.

7.1. Materials and Methods

All chemicals and supplies mentioned in these examples can be obtained from standard commercial sources unless otherwise indicated. Wortmannin can be obtained from Calbiochem (Cambridge, Mass.).

Synthesis of phenoxazine compounds of formula (I). The phenoxazine compounds of the invention can be prepared in pure form according to methods described in other publications. See, in particular, Horton et al. Mol. Pharmacol. 1993;44:552-559; Eregowda et al. Indian J. Chem. 2000;39B:243-259; and Eregowda et al. Asian J. Chem. 1999; 11:878-905. Each phenoxazine compound is preferably dissolved in dimethylsulfoxide (DMSO) before adding it to cell culture medium (final concentration 0.1%).

Synthesis of acridone compounds of formula (III). The acridone compounds of the invention can be prepared as follows: Acridones of formula (III) wherein K is alkoxy can be prepared in pure form according to methods previously described, for example, by Hegde et al. Eur. J. Med. Chem. 2004;39:161-177. Acridones of formula (III) wherein J is alkoxy can be prepared in pure form according to methods previously described, for example, by Krishnegowda et al. Biorg. Med. Chem. 2002;10:2367-2380.

The novel acridones of formula (III) wherein J is halogen may be generated synthetically, for example, as described below. Each acridone compound is preferably dissolved in dimethylsulfoxide (DMSO) before adding it to cell culture medium (final concentration 0.1%).

Synthesis of 2-Chloroacridone

Preparation of 4′-Chlorodiphenylamine-2-carboxylic acid by Ullmann Condensation. To a mixture of o-Chlorobenzoic acid (10 g, 0.064 mol), p-Chloroaniline (8.1 g, 0.064 mol) and copper powder (0.2 g) in 60 mL of isoamylalcohol, dry K₂CO₃ (10 g) was slowly added and the contents were refluxed for 6 h. The isoamylalcohol was removed by steam distillation and the mixture poured into 1 L of hot water and acidified. Precipitate formed was filtered, washed with hot water and collected. The crude acid was dissolved in aqueous sodium hydroxide solution, boiled in the presence of activated charcoal and filtered. On acidification, light yellowish precipitate was obtained which was washed with hot water and recrystallized from aqueous methanol to give a light yellow solid 4′-Chlorodiphenylamine-2-Carboxylic acid (yield 13.4 g, 84%, mp 186° C.).

Cyclization of 4′-Chlorodiphenylamine-2-carboxylic acid to 2-Chloroacridone. 4′-Chlorodiphenylamine-2-Carboxylic acid (10 g,) was taken in a flask to which was added 100 g of polyphosphoric acid. The reaction mixture was heated on a water bath at 100° C. for 3 h with stirring. Appearance of yellow color indicated the completion of the reaction. Then, it was poured into 1 L of hot water and made alkaline by liquor ammonia. The yellow precipitate that formed was filtered, washed with hot water and collected. The sample of 9 (10-H)-2-Chloroacridone was recrystallized from acetic acid (yield 7.41 g, 80%, mp 398° C.). UV λₘₐₓ (ε) (MeOH): 214 (23,135), 257 (43,240), 299 (2616), 386 (7482) nm. IR: 3655, 2987, 2855, 1627, 1164, 960, 754, 683 cm⁻¹. ¹H-NMR (DMSO-d₆): δ 7.18-8.21 (m, Ar—H, 7H, H₁, H₃, H₄ and H₅-H₈), and 12.06 (s, N—H). ¹³C-NMR (DMSO-d₆): δ 127.92 (C₁), 113.1(C₂), 126.00(C₃), 120.09 (C₄), 117.63(C₅), 135.95(C₆), 121.2(C₇), 133.82(C₈), 175.64(C₉), 113.08(C_{9′}), 139.77(C_{4′}), 140.79(C_{10′}) and 121.75 (C_{8′}). MS: m/z (%) 231 [(M+H)⁺, 100]. Anal. (C₁₃H₈NOCl) C, H, N.

Synthesis of N¹⁰-Alkylated 2-Chloroacridones via Phase Transfer Catalysis

10-(3′-Chloropropyl)-2-chloroacridone (Compound 6). 2-Chloroacridone (6 g, 0.026 mol) was dissolved in tetrahydrofuran (100 mL), and then 6N potassium hydroxide (50 mL) and tetrabutylammonium bromide (2 g, 0.006 mol) were added to it. This mixture was then stirred at room temperature for 30 min and. Next 1-bromo-3-chloropropane (0.065 mol) was slowly added into the reaction mixture, and the mixture stirred for an additional 48 h at room temperature. Tetrahydrofuran was evaporated and the aqueous layer extracted with chloroform. The chloroform layer was washed with water, dried over anhydrous sodium sulfate and rotavaporated. The crude product was purified by column chromatography to give a yellow solid of 10-(3′-Chloropropyl)-2-chloroacridone (yield 6.2 g, 52%, mp 141° C.). UV λₘₐₓ (ε) (MeOH): 214 (22,819), 254 (36,633), 299 (2491), 399(6732) nm. IR: 2940, 1628, 1460, 1044, 961, 753, 682 cm⁻¹. ¹H-NMR (DMSO-d₆): δ 7.36-8.38 (m, Ar—H, 7H, H₁, H₃, H₄ and H₅—H₈), 3.78-3.81 (t, 2H, Hₖ), 3.85-3.91 (t, 2H, Hₘ), and 2.32-2.5 (m, 2H, H₁). ¹³C-NMR (DMSO-d₆): δ 128.97 (C₁), 121.52 (C₂), 126.91 (C₃), 114.62 (C₄), 113.80 (C₅), 136.19(C₆), 121.31(C₇), 134.09(C₈), 175.76 (C₉), 122.85(C_{9′}), 139.99(C_{4′}), 141.01(C_{10′}), 116.97 (C_{8′}), 44.08 (Cₖ), 30.19(Cₗ) and 41.85(Cₘ). MS: m/z (%) 308 [(M+H)⁺, 100]. Anal. (C₁₆H₁₃NOCl₂) C, H, N.

10-(3′-N-Diethylaminopropyl)-2-chloroacridone (Compound 1). To the solution of 10-(3′-Chloropropyl)-2-chloroacridone (1.12 g, 3.66 mmol) in 60-mL of acetonitrile, 1.57 g KI and 2.54 g K₂CO₃ were added and the mixture stirred at reflux conditions for 30 min. Then, diethylamine (1.17 g, 16.02 mmol) was added slowly. The reaction mixture was refluxed for 18 h, cooled to room temperature and extracted with chloroform. The chloroform layer was washed with water thrice, dried over anhydrous sodium sulfate and rotavaporated. The product was purified by column chromatography to give a yellow oily product which was converted into hydrochloride salt of 10-(3′-N-Diethylaminopropyl)-2-chloroacridone (yield 0.55 g, 40%, mp 110-112° C.). UV λₘₐₓ (ε) (MeOH): 218 (15,250), 255 (27,850), 391 (5,200), 410 (5800) nm. IR: 3504, 2919, 1724, 1591, 1263, 948, 753, 673, 544 cm⁻¹. ¹H-NMR (DMSO-d₆): δ 7.35-8.34 (m, Ar—H, 7H, H₁, H₃, H₄ and H₅-H₈), 3.09-3.81 (m, 8H, Hₖ, Hₘ, Hₐ, H_{b}), 2.08-2.5 (t, 2H, Hₑ) and 1.23-1.27 (m, 6H, H_{c} and H_{d}). ¹³C-NMR (DMSO-d₆): δ 128.38(C₁), 121.89 (C₂), 126.64(C₃), 116.01(C₄), 113.74(C₅), 136.61(C₆), 121.87(C₇), 134.82(C₈), 175.74(C₉), 122.96(C_{9′}), 140.84(C_{4′}), 141.22(C_{10′}), 118.80 (C_{8′}), 58.87(Cₖ), 22.94(Cₗ), 24.42 (Cₘ), 51.61(Cₐ and C_{b}) and 13.33 (C_{c} and C_{d}). MS: m/z (%) 346 [(M+H)⁺, 100]. Anal. (C₂₀H₂₄N₂OCl 2) C, H, N.

10-[3′-N-(Methylpiperazino)propyl]-2-chloroacridone (Compound 2). The experimental procedure used for 10-(3′-N-Diethylaminopropyl)-2-chloroacridone is applicable with 1.25 g (4.08 mmol) of 2, 1.76 g of KI, 2.86 g of K₂CO₃ and 1.38 g (13.7 mmol) of N-methylpiperazine. The oily residue was purified by column chromatography and converted into hydrochloride salt of 10-[3′-N-(Methylpiperazino)propyl]-2-chloroacridone (yield 0.8 g, 42%, mp 268° C.). UV λₘₐₓ (ε) (MeOH): 217 (19,993), 256 (58,980), 394 (15,245), 412 (16,744) nm. IR: 3399, 2958, 1610, 1494, 1267, 1058, 958, 755, 685 cm⁻¹. ¹H-NMR (DMSO-d₆): δ 7.26-8.34 (m, Ar—H, 7H, H₁, H₃, H₄ and H₅-H₈), 2.08-3.77 (m, 12H, Hₖ, Hₘ, Hₐ, H_{b}, H_{c} and H_{d}), 2.5 (s, 3H, H_{c}) and 1.87-2.27(m, 2H, Hₗ). ¹³C-NMR (DMSO-d₆): δ 128.68 (C₁), 121.29 (C₂), 126.57 (C₃), 114.19 (C₄), 113.58(C₅), 136.31(C₆), 121.30(C₇), 134.93(C₈), 175.38(C₉), 122.36(C_{9′}), 139.34(C_{4′}), 141.36(C_{10′}), 116.39 (C_{8′}), 44.16 (Cₖ), 23.09(Cₗ), 42.49(Cₘ), 50.09(Cₐ and C_{b}), 51.45(C_{c} and C_{d}) and 27.58(Cₑ). MS: m/z (%) 371 [(M+H)⁺, 100].

10-(3′-N-Piperidinopropyl)-2-chloroacridone (Compound 3). The procedure used for 10-(3′-N-Diethylaminopropyl)-2-chloroacridone was repeated with 1.2 g (3.92 mmol) of 10-(3′-Chloropropyl)-2-chloroacridone, 1.75 g of KI, 2.74 g of K₂CO₃, and 1.25 g (14.82 mmol) of piperidine. The purified product was converted into the hydrochloride salt (yield 0.75 g, 49%, mp 246-250° C.). UV λₘₐₓ (ε) (MeOH): 216 (24,357), 255 (37,928), 382 (5,964), 400 (6904) nm. IR: 3384, 2982, 1625, 1465, 958, 754, 663 cm⁻¹. ¹H-NMR (DMSO-d₆): δ 7.34-8.33 (m, Ar—H, 7H, H₁, H₃, H₄ and H₅-H₈), 3.31-3.41 (m, 8H, Hₖ, Hₘ, Hₐ, H_{b}), 2.25-2.89 (m, 2Hₗ, H_{c} and H_{d}), and 1.69-1.84 (m, 3H, Hₑ). ¹³C-NMR (DMSO-d₆): δ 126.75 (C₁), 122.35 (C₂), 125.31(C₃), 118.68 (C₄), 116.04 (C₅), 134.81(C₆), 121.94(C₇), 133.99(C₈), 175.61(C₉), 126.06(C_{9′}), 139.90(C_{4′}), 141.15 (C_{10′}), 121.35 (C_{8′}), 52.90 (Cₖ), 21.39 (Cₗ), 42.98 (Cₘ), 52.65 (Cₐ and C_{b}), 22.37 (C_{c} and C_{d}) and 21.21(Cₑ). MS: m/z (%) 356 [(M+H)⁺, 100]. Anal. (C₂₁H₂₄N₂OCl₂) C, H, N.

10-(3′-N—I(P-Hydroxyethyl)piperazino]propyl)-2-chloroacridone. The method employed for 10-(3′-N-Diethylaminopropyl)-2-chloroacridone was used with 1.0 g (3.26 mmol) of 10-(3′-Chloropropyl)-2-chloroacridone, 1.41 g of KI, 2.28 g of K₂CO₃ and 2.06 g (15.8 mmol, 1.94 mL) of (β-hydroxyethyl)piperazine. The oily residue was purified by column chromatography and was converted into hydrochloride salt of 10-(3′-N-[(β-Hydroxyethyl)piperazino]propyl)-2-chloroacridone (yield 0.62 g, 40%, mp 260-262° C.). UV λₘₐₓ (ε) (MeOH): 216 (18,600), 255 (54,068), 386 (8851), 404 (10,310) nm. IR: 3368, 2958, 1611, 1560, 1459, 1270, 961, 758, 685 cm⁻¹. ¹H-NMR(DMSO-d₆): δ7.15-8.34 (m, Ar—H, 7H, H, H₃, H₄ and H₅-H₈), 4.55(s, —OH), 3.26-3.6 (m, 12H, Hₑ, Hₘ, Hₐ, H_{b}, H_{c}, H_{d}), 3.82 (m, 2H, Hk), 4.46 (m, 2H, H_{f}) and 1.38-1.4 (m, 4H, Hₗ, Hₘ). ¹³C-NMR (DMSO-d₆): δ 128.57 (C₁), 122.79 (C₂), 126.82(C₃), 115.98 (C₄), 113.95 (C₅), 136.73(C₆), 121.41(C₇), 134.97 (C₈), 175.55 (C₉), 121.76(C_{9′}), 140.22(C_{4′}), 141.26(C_{10′}), 118.75 (C_{8′}), 52.47 (Cₖ), 21.57 (Cₗ), 42.70 (Cₘ), 47.88 (Cₐ and C_{b}), 48.30 (C_{c} and C_{d}), 54.84(Cₑ) and 57.37(C_{f}). MS:m/z (%) 401 [(M+H)⁺, 100]. Anal. (C₂₂H₂₈N₃O₂Cl₃) C, H, N.

10-[3-N-Pyrrolidinopropyl]-2-chloroacridone (Compound 4). Amounts of 1.02 g (3.33 mmol) of 10-(3′-Chloropropyl)-2-chloroacridone, 1.65 g of KI, 2.64 g of K₂CO₃ and 0.88 g (0.8 mL, 8.34 mmol) of piperidine were refluxed and processed according to the procedure used for 10-(3′-N-Diethylaminopropyl)-2-chloroacridone. The crude product was purified by column chromatography and the pale yellow oily product was converted into hydrochloride salt of 10-[3′-N-Pyrrolidinopropyl]-2-chloroacridone (yield 0.65 g, 48%, mp 183-185° C.). UV λₘₐₓ (ε) (MeOH): 217 (15,324), 256 (31,465), 386 (5183), 405 (5972) nm. IR: 3393, 2947, 1621, 1492, 1457, 1267, 961, 758, 682 cm⁻¹. ¹H-NMR (DMSO-d₆): δ 7.38-8.40 (m, Ar—H, 7H, H₁, H₃, H₄ and H₅-H₈), 3.14-3.94 (m, 8H, Hₖ, Hₘ, Hₐ, H_{b}), and 1.56-2.5(m, 2Hₗ, H_{c} and H_{d}). ¹³C-NMR (DMSO-d₆): δ 128.60 (C₁), 122.83 (C₂), 126.87(C₃), 120.12 (C₄), 118.73(C₅), 136.74(C₆), 122.12 (C₇), 134.97 (C₈), 175.34(C₉), 127.88(C_{9′}), 140.24(C_{4′}), 141.18(C_{10′}), 121.46 (C_{8′}), 57.53 (Cₖ), 23.46(C₁), 51.26(Cₘ), 53.33(Cₐ and C_{b}) and 22.90(C_{c} and C_{d}). MS: m/z (%) 342 [(M+H)⁺, 100].

10-(3′-N-Morpholinopropyl)-2-chloroacridone (Compound 5). The hydrochloride salt of 10-(3′-N-Morpholinopropyl)-2-chloroacridone (yield 0.6 g, 43%, mp 248-250° C.) was obtained by following the procedure of 10-(3′-N-Diethylaminopropyl)-2-chloroacridone with 1.1 g of 10-(3′-Chloropropyl)-2-chloroacridone (3.59 mmol), 1.55 g KI, 2.5 g of K₂CO₃ and 1.17 g (13.4 mmol) of morpholine. UV λₘₐₓ (ε) (MeOH): 217 (23,445), 256(54,741), 389 (7,075), 408 (11,260) nm. IR: 3429, 2869, 1617, 1494, 1272, 874, 684 cm⁻¹. ¹H-NMR (DMSO-d₆): δ 7.36-8.35 (m, Ar—H, 7H, H₁, H₃, H₄ and H₅-H₈), 3.04-3.10 (t, 4H, H_{c} and H_{d}), 2.27 (m, 4H, Hₖ, Hₘ), 2.50 (t, 4H, Hₐ, H_{b}), and 1.21-1.97 (m, 2H, Hₗ). ¹³C-NMR (DMSO-d₆): δ 126.77(C₁), 122.34 (C₂), 125.33(C₃), 118.15 (C₄), 115.64(C₅), 135.32(C₆), 121.99(C₇), 134.47 (C₈), 176.34 (C₉), 126.52(C_{9′}), 139.72 (C_{4′}), 141.00(C_{10′}), 121.08 (C_{8′}), 53.08 (Cₖ), 21.12 (Cₗ), 42.57(Cₘ), 51.39 (Cₐ and C_{b}) and 63.27 (C_{c} and C_{d}). MS: m/z (%) 358 [(M+H)⁺, 100]. Anal.(C₂₀H₂₂N₂O₂Cl₂) C, H, N.

10-(3′-N-[Bis[hydroxyethyl]amino]propyl)-2-chloroacridone. The experimental steps used for 10-(3′-N-Diethylaminopropyl)-2-chloroacridone were repeated with 1 g (3.26 mmol) of 10-(3′-Chloropropyl)-2-chloroacridone, 1.48 g of KI, 2.3 g of K₂CO₃ and 0.88 g (8.34 mmol) of N,N-diethanolamine. The crude product was purified by column chromatography to give a light yellow solid 10-(3′-N-[Bis[hydroxyethyl]amino]propyl)-2-chloroacridone (yield 0.65 g, 48%, mp 148-150° C.). UV λₘₐₓ (ε) (MeOH): 216 (30,000), 255 (61,447), 386 (9659), 402 (10,681) nm. IR: 3286, 2973, 2885, 1630, 1488, 1267, 960, 757, 682 cm⁻¹. ¹H-NMR (DMSO-d₆): δ 7.35-8.34 (m, Ar—H, 7H, H₁ H₃, H₄ and H₅-H₈), 3.25-3.28 (t, 4H, Hₖ, Hₘ), 3.53-3.79(t, 8H, Hₐ, H_{b}), 3.81-3.92 (m, 4H, H_{c} and H_{d}), 2.5 (s, 2H, Hₑ and H_{f}, disappearing on D₂O exchange), and 2.07-2.08 (q, 2H, H₁). ¹³C-NMR (DMSO-d₆): δ 128.38 (C₁), 122.55 (C₂), 126.65(C₃), 116.04 (C₄), 113.83(C₅), 136.50(C₆), 121.23(C₇), 134.69 (C₈), 175.36(C₉), 121.88(C_{9′}), 140.32(C_{4′}), 141.22(C_{10′}), 118.80(C₈), 47.36(Cₖ), 21.09(Cₗ), 42.78(Cₘ), 46.22(Cₐ and C_{b}) and 8.52(C_{c} and C_{d}). MS: m/z (%) 376 [(M+H)⁺, 100]. Anal.(C₂₀H₂₃N₂O₃Cl) C, H, N.

10-(4′-Chlorobutyl)-2-chloroacridone (Compound 13). Yellow crystals of 10-(4-Chlorobutyl)-2-chloroacridone in the pure form (yield 6.5 g, 55%, mp 101-106° C.) were prepared by following the procedure used for 10-(3′-Chloropropyl)-2-chloroacridone with 6 g (0.026 mol) of 2-Chloroacridone and 1-bromo-4-chlorobutane (0.065 mmol). UV λₘₐₓ (ε) (MeOH): 217 (15,914), 254 (31,930), 392 (8,004), 412 (8,687) nm. IR: 3395, 2928, 1614, 1591, 1256, 965, 752 cm⁻¹. ¹H-NMR (DMSO-d₆): δ 7.25-8.3 (m, Ar—H, 7H, H₁, H₃, H₄ and H₅-H₈), 3.61-3.73(t, 4H, Hₖ, Hₘ), and 1.86-3.34 (m, 4H, Hₗ, Hₘ). ¹³C-NMR (DMSO-d₆): δ 126.75(C₁), 122.51(C₂), 125.40(C₃), 118.47 (C₄), 115.95(C₅), 134.48(C₆), 121.64(C₇), 133.75 (C₈), 175.37(C₉), 125.86(C_{9′}), 140.03(C_{4′}), 141.29 (C_{10′}), 117.67 (C_{8′}), 44.70 (Cₖ), 25.51 (Cₗ), 29.26(Cₘ) and 44.96(Cₙ). MS: m/z (%) 320 [(M+H)⁺, 100]. Anal.(C₁₇H₁₅NOCl₂) C, H, N.

10-(4′-N-Diethylaminobutyl)-2-chloroacridone (Compound 7). The procedure used for 10-(3′-N-Diethylaminopropyl)-2-chloroacridone was followed with 1.2 g (3.8 mmol) of 10-(4′-Chlorobutyl)-2-chloroacridone, 1.57 g of KI, 2.64 g of K₂CO₃ and 1.3 g (17.8 mmol) of N,N-diethylamine. The product was purified by column chromatography to give a yellow oily product which was converted into hydrochloride salt of 10-(4′-N-Diethylaminobutyl)-2-chloroacridone (yield 0.73 g, 50%, mp 100-104° C.). UV λₘₐₓ (O) (MeOH): 216 (23,266), 255 (36,367), 392 (6,864) nm. IR: 3386, 2941, 1625, 1458, 1276, 960, 756 cm⁻¹. ¹H-NMR (DMSO-d₆): δ 7.35-8.35 (m, Ar—H, 7H, H₁, H₃, H₄ and H₅-H₈), 4.48-4.52(t, 2H, Hₖ), 3.08-3.81 (m, 8H, Hₘ, Hₐ, H_{b}), 1.22-1.26 (m, 6H, H_{c} and H_{d}) and 1.83-2.5 (t, 4H, H_{l, H}ₙ). ¹³C-NMR (DMSO-d₆): δ 126.80 (C₁), 122.57 (C₂), 125.41(C₃), 118.76(C₄), 116.16(C₅), 134.60(C₆), 121.78(C₇), 133.83(C₈), 175.46(C₉), 125.93(C_{9′}), 140.13(C_{4′}), 141.37(C_{10′}), 121.55 (C_{8′}), 54.76 (Cₖ), 17.56(Cₗ), 24.20(Cₘ), 45.15(Cₙ), 50.41(Cₐ and C_{b}) and 8.50(C_{c} and C_{d}). MS: m/z (%) 358 [(M+H)⁺, 100]. Anal.(C₂₁H₂₆N₂OCl₂) C, H, N.

10-(4′-N-(Methylpiperazino) butyl)-2-chloroacridone (Compound 8). Amounts of 1.1 g of 10-(4′-Chlorobutyl)-2-chloroacridone (3.43 mmol), 1.42 g of KI, 2.37 g of K₂CO₃ and 1.56 g (15.6 mmol) of N-methylpiperazine were refluxed and processed according to the procedure used for 10-(4′-N-Diethylaminobutyl)-2-chloroacridone. The crude product was chromatographed on silica gel to get the pure base which was then converted into hydrochloride salt of 10-(4′-N-(Methylpiperazino)butyl)-2-chloroacridone (yield 0.8 g, 57%, mp 260-262° C.). UV λₘₐₓ (ε) (MeOH): 216 (18,164), 256 (59,249), 392 (16,542), 412 (18,380) nm. IR: 3445, 2829, 1716, 1634, 1480, 1253, 962, 754, 652 cm⁻¹. ¹H-NMR (DMSO-d₆): δ 7.34-8.32 (m, Ar—H, 7H, H₁, H₃, H₄ and H₅-H₈), 2.8-3.7 (m, 12H, Hₖ, Hₘ, Hₐ, H_{b}, H_{c}, H_{d}), 2.5 (s, 3H, Hₑ) and 1.84-2.5(m, 4H, Hₗ, Hₘ). ¹³C-NMR (DMSO-d₆): δ 126.77 (C₁), 122.51 (C₂), 125.37(C₃), 118.73 (C₄), 116.12(C₅), 134.61(C₆), 121.76(C₇), 133.84 (C₈), 175.44(C₉), 125.90(C_{9′}), 140.06(C_{4′}), 141.31(C_{10′}), 121.51 (C_{8′}), 55.20 (Cₖ), 20.23 (Cₗ), 20.06(Cₘ), 45.16(Cₙ), 49.43(Cₐ and C_{b}), 48.05(C_{c} and C_{d}) and 42.02(Cₑ). MS: m/z (%) 385 [(M+H)⁺, 100].

10-(4′-N-Piperidinobutyl)-2-chloroacridone (Compound 9). Compound 10-(4-Chlorobutyl)-2-chloroacridone (1.5 g, 4.68 mmol), KI (1.94 g), K₂CO₃ (3.23 g) and piperidine (1.4 g, 23.42 mmol) were used for this reaction and the rest of the steps used for 10-(4′-N-(Methylpiperazino) butyl)-2-chloroacridone remain the same. The oily residue was then converted into hydrochloride salt of 10-(4′-N-Piperidinobutyl)-2-chloroacridone (yield 1 g, 70%, mp 199-200° C.). UV λₘₐₓ (ε) (MeOH): 217 (15,900), 259 (36,500), 393 (8,367), 411 (9,400) nm. IR:3400, 2880, 1629, 1595, 1459, 1263, 959, 758, 682 cm⁻¹. ¹H-NMR (DMSO-d₆): δ 7.36-8.36 (m, Ar—H, 7H, H₁, H₃, H₄ and H₅-H₈), 3.31-3.41 (m, 8H, Hₖ, Hₘ), 1.38-1.40(m, 8H, Hₐ, H_{b}, H_{c} and H_{d}). 1.68-1.96 (m, 2H, Hₑ) and 2.81-2.84(q, 4H, Hₙ, Hₗ). ¹³C-NMR (DMSO-d₆): δ 126.84 (C₁), 122.57 (C₂), 125.44(C₃), 118.72 (C₄), 116.12(C₅), 134.62(C₆), 121.80(C₇), 133.86 (C₈), 175.41(C₉), 125.95(C_{9′}), 140.14(C_{4′}), 141.37(C_{10′}), 121.58 (C_{8′}), 55.44 (Cₖ), 20.35 (Cₗ), 24.32(Cₘ), 45.04(Cₙ), 49.43(Cₐ and C_{b}) and 48.05(C_{c} and C_{d}). MS: m/z (%) 370 [(M+H)⁺, 100]. Anal.(C₂₂H₂₆N₂OCl₂) C, H, N.

10-(4′-N-[(β-Hydroxyethyl)piperazino]butyl)-2-chloroacridone (Compound 10). The procedure used for 10-(3′-N-[(β-Hydroxyethyl)piperazino]propyl)-2-chloroacridone was repeated with 1.1 g (3.43 mmol) of 10-(4′-Chlorobutyl)-2-chloroacridone, 1.42 g of KI, 2.37 g of K₂CO₃ and 1.56 g (14.6 mmol, 1.5 mL) of (β-hydroxyethyl)piperazine. The oily residue was purified by column chromatography and dissolved in anhydrous acetone and treated with ethereal hydrochloride to give hydrochloride salt of 10-(4′-N-[(β-Hydroxyethyl)piperazino]butyl)-2-chloroacridone (yield 0.62 g, 40%, mp 260-262° C.). UV λₘₐₓ (ε) (MeOH): 216 (25,436), 260 (29,499), 393 (5,526), 410 (6203) nm. IR: 3509, 2940, 1728, 1480, 1255, 959, 758, 696 cm⁻¹. ¹H-NMR (DMSO-d₆): δ7.31-8.3 (m, Ar—H, 7H, H₁, H₃, H₄ and H₅-H₈), 4.64 (s, 1H, Hg), 2.49-3.26 (m, 12H, Hₑ, Hₘ, Hₐ, H_{b}, H_{c}, H_{d}), 3.82 (m, 2H, Hₖ), 4.46 (m, 2H, H_{f}) and 1.38-1.4 (m, 4H, Hₗ, Hₘ). ¹³C-NMR (DMSO-d₆): δ 126.74 (C₁), 122.47 (C₂), 124.65(C₃), 118.71 (C₄), 116.11(C₅), 134.60(C₆), 121.48(C₇), 133.83 (C₈), 175.43 (C₉), 125.89(C_{9′}), 140.03(C_{4′}), 141.28 (C_{10′}), 121.48 (C_{8′}), 56.37 (Cₖ), 20.25 (Cₗ), 24.06(Cₘ), 45.17(Cₙ), 47.91(Cₐ and C_{b}), 45.71(C_{c} and C_{d}) and 55.33(Cₑ). MS: m/z (%) 413 [(M+H)⁺, 100]. Anal.(C₂₃H₃₀N₃O₂Cl₃) C, H, N.

10-[4′-N-Pyrrolidinobutyl]-2-chloroacridone (Compound 11). The procedure employed for 10-[3′-N-Pyrrolidinopropyl]-2-chloroacridone was used with 0.85 g (2.65 mmol) of 10-(4′-Chlorobutyl)-2-chloroacridone, 1.1 g of KI, 1.85 g of K₂CO₃ and 0.942 g (1.1 mL, 13.25 mmol) of pyrrolidine. The crude product was purified by column chromatography to give a pale yellow oily product, which was then converted into hydrochloride salt of 10-[4′-N-Pyrrolidinobutyl]-2-chloroacridone (yield 0.8 g, 57%, mp 268-271° C.). UV λₘₐₓ (ε) (MeOH): 205 (10,784), 222 (16,995), 256 (57,617), 398 (8,187) nm. IR: 3460, 2951, 1654, 1428, 1248, 962, 686 cm⁻¹. ¹H-NMR (DMSO-d₆): δ 7.36-8.36 (m, Ar—H, 7H, H₁, H₃, H₄ and H₅-H₈), 3.31-3.41 (m, 8H, Hₖ, Hₘ), 1.38-1.40 (m, 8H, Hₐ, H_{b}, H_{c} and H_{d}) and 2.81-2.84(q, 4H, Hₙ, Hₗ). ¹³C-NMR (DMSO-d₆): δ 126.77 (C₁), 122.50 (C₂), 125.36(C₃), 118.79 (C₄), 116.16(C₅), 134.65(C₆), 121.78(C₇), 133.88 (C₈), 175.46(C₉), 125.90(C_{9′}), 140.07(C_{4′}), 141.32 (C_{10′}), 121.51 (C_{8′}), 53.61 (Cₖ), 17.56 (Cₗ), 22.83(Cₘ), 45.15(Cₙ), 50.41(Cₐ and C_{b}) and 24.20(C_{c} and C_{d}). MS: m/z (%) 370 [(M+H)⁺, 100]. Anal.(C₂₁H₂₄N₂OCl₂) C, H, N.

10-(4′-N-Morpholinobutyl)-2-chloroacridone (Compound 12). The procedure used for 10-(3′-N-Morpholinopropyl)-2-chloroacridone was repeated with 0.9 g (2.81 mmol) of 10-(4′-Chlorobutyl)-2-chloroacridone, 1.16 g of KI, 2.5 g of K₂CO₃ and 0.98 g (11.24 mmol) of morpholine to get an oily product, which was purified by column chromatography. Light colored oil thus obtained was converted into hydrochloride salt of 10-(4′-N-Morpholinobutyl)-2-chloroacridone (yield 0.65 g, 56%, mp 238-240° C.). UV λₘₐₓ (ε) (MeOH): 203 (18,088), 222 (16,995), 256 (57,617), 398 (8,187) nm. IR: 3397, 2966, 1610, 1261, 971, 756 cm⁻¹. ¹H-NMR (DMSO-d₆): δ 7.3-8.34 (m, Ar—H, 7H, H₁, H₃, H₄ and H₅-H₁₈), 3.14-3.10 (t, 4H, H_{c} and H_{d}), 2.37 (m, 4H, Hₖ, Hₘ), 2.50 (t, 4H, Hₐ, H_{b}), and 1.29-1.91 (m, 2H, Hₗ). ¹³C-NMR (DMSO-d₆): δ 128.49 (C₁), 122.65 (C₂), 121.99(C₃), 115.99(C₄), 113.83(C₅), 136.72 (C₆), 121.31(C₇), 134.93 (C₈), 175.68(C₉), 125.90(C_{9′}), 140.06(C_{4′}), 141.31 (C_{10′}), 121.51 (C_{8′}), 55.80 (Cₖ), 23.84 (Cₗ), 20.04(Cₘ), 44.97(Cₙ), 51.17(Cₐ and C_{b}) and 63.21(C_{c} and C_{d}). MS: m/z (%) 372 [(M+H)⁺, 100].

10-(4′-N-[Bis[hydroxyethyl]amino]butyl)-2-chloroacridone. The procedure used for 10-(3′-N-[Bis[hydroxyethyl]amino]propyl)-2-chloroacridone was followed with 1 g (3.12 mmol) of 10-(4′-Chlorobutyl)-2-chloroacridone, 1.3 g of KI, 2.2 g of K₂CO₃ and 1 g (9.58 mmol) of diethanolamine. The product was purified by column chromatography to give a pale yellow solid 10-(4′-N-[Bis[hydroxyethyl]amino]butyl)-2-chloroacridone (yield 0.7 g, 53%, mp 243° C.). UV λₘₐₓ (ε) (MeOH): 217 (8,626), 255 (24,792), 392 (6,942), 412 (7,650) nm. IR: 3422, 2958, 1629, 1496, 1455, 1254, 971, 756, 682 cm⁻¹. ¹H-NMR (DMSO-d₆): δ 7.34-8.34 (m, Ar—H, 7H, H₁, H₃, H₄ and H₅-H₈), 3.40-3.49 (t, 4H, Hₖ, Hₘ), 2.08-2.57(m, 4H, H₁, Hₐ, H_{b}), 1.65-1.9(m, 4H₁, H_{c} and H_{d}) and 2.5 (s, 2H, Hₑ and H_{f}, disappearing on D₂O exchange). ¹³C-NMR (DMSO-d₆): δ 126.74 (C₁), 122.49 (C₂), 125.33(C₃), 118.79 (C₄), 116.17 (C₅), 134.55 (C₆), 121.65 (C₇), 133.82 (C₈), 175.47 (C₉), 125.82(C_{9′}), 140.13(C_{4′}), 141.39 (C_{10′}), 121.55 (C_{8′}), 59.33 (Cₖ), 23.67 (Cₗ), 24.24 (Cₘ), 45.60 (Cₙ), 56.55(Cₐ and C_{b}) and 59.33 (C_{c} and C_{d}). MS: m/z (%) 390 [(M+H)⁺, 100]. Anal.(C₂₁H₂₅N₂O₃Cl) C, H, N.

Synthesis of N¹⁰-Alkylated 2-Bromoacridones via Phase Transfer Catalysis

The corresponding 2-bromoacridones may be synthesized as described for the individual 2-chloroacridone compounds above, except that the starting materials are o-chlorobenzoic acid and p-bromoaniline.

Cell lines and growth conditions. The human cell lines Rh1, Rh18, and Rh30 (ATCC Deposit # CRL 2061) have been described, e.g., by Hazelton et al. Cancer Res 1987;47:4501-4507 and Hosoi et al. Cancer Res. 1999;59:886-894. Rh1, Rh18 and Rh30 cells can each be grown in antibiotic free RPMI-1640 medium (available from BioWhittaker, Walkersville, Md.), supplemented with 10% fetal bovine serum (available from HyClone Laboratories, Logan, Utah) and 2 mM L-glutamine (available from BioWhittaker, Walkersville, Md.) at 37° C. in an atmosphere of 5% CO₂. For serum free experiments, cells can be cultured in modified N2E (MN2E) medium (DMEM/F-12, 1:1 mixture) (Sigma, St. Louis, Mo.) supplemented with 1 μg/ml human holo transferrin, 30 nM sodium selenite, 20 nM progesterone, 100 μM putrescine, 30 nM vitamin E phosphate, and 50 μM ethanolamine. Cells in MN2E medium containing 5 μg/ml bovine fibronectin (available from Sigma, St. Louis, Mo.) are preferably plated, and allowed to attach overnight at 37° C. in a humidified, 5% CO₂ atmosphere.

Cellular screening for inhibitors. Rh1, Rh18 and Rh30 cells can each be seeded at a density of 4×10⁶/10-cm plate in serum-free medium for overnight attachment. The cells can then be exposed to 0.1% DMSO or to a test compound (for example, a phenoxazine or acridone compound) for one hour, then stimulated with Insulin-like growth factor-I (IGF-I) (10 ng/ml) for 10 minutes.

Western blot analysis. Cells are rapidly washed with ice-cold phosphate-buffered saline (PBS), placed on ice, and lysed in mammalian protein extraction reagent (M-PER; available from Pierce, Rockford, Ill.) containing one Complete™ mini protease inhibitor tablet (available from Boehringer Mannheim, Mannheim, Germany), 1 mM phenylmethylsulfonyl fluoride, 1 mM Na₃VO₄ and 1 mM NaF. Cellular debris is pelleted by centrifugation at 17,500×g for 10 minutes at 4° C. The protein concentration of the supernatants is measured by the bicinchoninic acid assay (e.g., using the BCA™ Protein Assay Kit, Pierce, Rockford, Ill., catalog number 23225 or 23227) using bovine serum albumin as the standard.

For the analysis of AKT, ERK-1/2, mTOR, p70S6 kinase, ribosomal protein S6 (rpS6 or S6), and glycogen synthase kinase 3 (GSK-3), equivalent amounts of protein can be separated on a 12% SDS-polyacrylamide gel (available from BioRad, Hercules, Calif.) by electrophoresis and subsequently transferred to a nitrocellulose membrane (also available from BioRad). After a 1 hour incubation in 1×TBS containing 0.05% Tween 20 and 5% blocking reagent (skim milk) (available from Upstate Biotechnology, Lake Placid, N.Y.) at room temperature, the wet nitrocellulose membranes are incubated with appropriate antibodies (available from Cell Signaling Technology, Beverly, Mass.): rabbit polyclonal antiserum specific for the phosphorylated Ser473 or Thr308 of AKT (dilution 1:1000); rabbit polyclonal antiserum specific for phosphorylated Thr202/Tyr204 of ERK-1/2 (dilution 1:1000); rabbit polyclonal antiserum specific for phosphorylated Ser2448 or Ser2481 of mTOR (dilution 1:1000); rabbit polyclonal antiserum specific for phosphorylated Thr389 of p70S6 kinase (dilution 1:4000); rabbit polyclonal antiserum specific for phosphorylated Ser235/236 of rpS6 (dilution 1:1000); or rabbit polyclonal antiserum specific for phosphorylated Ser21/9 of GSK-3α/β (dilution 1:1000). Horseradish peroxidase-conjugated goat anti-rabbit IgG antibody (dilution 1:10,000) can be used as the secondary antibody. Immunoreactive protein can be visualized using Renaissance chemiluminescence reagent (available from Life Science Products Inc., Boston, Mass.).

To ensure that equivalent amounts of protein are loaded on each gel, immunoblots can be treated with stripping buffer (62.5 mM Tris-HCl, pH 6.7; 2% SDS; and 100 mM β-mercaptoethanol) for 30 minutes at 50° C. and then incubated with one of the appropriate antibodies: rabbit polyclonal antibody to AKT (dilution 1:1000; available from Cell Signaling Technology, Beverly, Mass.); mouse monoclonal antibody 26E3 to mTOR (dilution 1:500; available from Santa Cruz Biotechnology Inc., Santa Cruz, Calif.); or mouse monoclonal antibody to β-tubulin (dilution 1:2000; Sigma, St. Louis, Mo.). Horseradish peroxidase-conjugated goat anti-rabbit IgG antibody (dilution 1:10,000) can be used as the secondary antibody. Bound antibody can be detected using Renaissance chemiluminescence reagent (available from Life Science Products Inc., Boston, Mass.).

Determination of cellular AKT kinase activity. AKT kinase activity can be quantitated using a commercial assay kit (available from Cell Signaling Technology, Beverly, Mass.) according to the manufacturer's instructions. Specifically, Rh1 cells are seeded in serum-free medium at a density of 4×10⁶ per 10-cm plate. After 24 hours, cells are exposed to either DMSO (0.1%) or a test compound (e.g., a phenoxazine or acridone compound) at 5 μM for one hour. Cells are then stimulated with ±IGF-I (10 nm/ml) for 10 minutes and washed once with ice-cold PBS. Cells are lysed in 200 μl of ice-cold 1× lysis buffer (20 mM Tris, pH 7.5; 150 mM NaCl; 1 mM EDTA; 1 mM EGTA; 1% Triton X-100; 2.5 mM sodium pyrophosphate; 1 mM β-glycerol phosphate; 1 mM Na₃VO₄; 1 mM phenylmethylsulfonyl fluoride; and 1 mM leupeptin) and incubated for 10 minutes on ice. The cell lysates are then centrifuged for 10 minutes at 17,500×g at 4° C. Volumes of the supernatants are preferably adjusted so that each sample contains an equal amount of protein (150 μg). The supernatants are then incubated with immobilized (cross-linked) anti-AKT antibody (Cell Signaling Technology, Beverly, Mass., catalog # 9279) for 3 hours at 4° C. The immunoprecipitates are pelleted and washed twice in ice-cold cell lysis buffer, and twice in kinase buffer (25 mM Tris, pH 7.5; 5 mM α-glycerol phosphate; 2 mM dithiothreitol; 0.1 mM Na₃VO₄; and 10 mM MgCl₂). The pellets are suspended in 40 μl of kinase buffer containing 200 μM ATP and 1 μg of a GSK-3 fusion protein (Cell Signaling Technology, Beverly, Mass., catalog #9278). This fusion protein is made up of a GSK-3alpha/beta peptide sequence, corresponding to residues surrounding GSK-3alpha/beta residue Ser21/9 (amino acid sequence CGPKGPGRRGRRRTSSFAEG; SEQ ID NO: 11), fused to the N-terminus of paramyosin. After incubating the suspensions at 30° C. for 30 minutes, the reaction is terminated by the addition of 3×SDS sample buffer (187.5 mM Tris-HCl, pH 6.8; 6% SDS; 30% glycerol; 150 mM dithiothreitol; and 0.03% bromophenol blue). The samples are boiled for five minutes. The proteins are separated on a 12% SDS polyacrylamide gel and subsequently transferred to a nitrocellulose membrane. Membranes are preferably incubated with rabbit polyclonal anti-phospho-GSK-3α/β (Ser21/9) antibody (available from Cell Signaling Technology, Beverly, Mass., catalog # 9331).

In vitro inhibition of recombinant AKT. In vitro kinase assays can be performed using an active, recombinant, full length AKT1/PKBα protein (available from Upstate Biotechnology, Lake Placid, N.Y.) or with an active, recombinant AKT1/PKBα protein, referred to herein as AKT1ΔPH, that lacks the pleckstrin homology domain (also available from Upstate Biotechnology). 10 ng of the recombinant enzyme in 25 μl 1× kinase buffer (25 mM Tris, pH 7.5; 5 mM β-glycerol phosphate; 2 mM dithiothreitol; 0.1 mM Na₃VO₄; and 10 mM MgCl₂) is mixed with 2.5 μl of DMSO and a test compound (5 μM). Samples are incubated on ice for 1 hour, at which time 1 μg of GSK-3 fusion protein (Cell Signaling Technology, Beverly, Mass., catalog #9278) is added followed by ATP (200 μM) to each reaction mixture. After incubating the suspensions at 30° C. for 30 minutes, the reaction can be terminated by the addition of 3×SDS sample buffer (187.5 mM Tris-HCl, pH 6.8; 6% SDS; 30% glycerol; 150 mM dithiothreitol; and 0.03% bromophenol blue). The samples are then boiled for five minutes. The proteins can be separated on a 12% SDS polyacrylamide gel, and subsequently transferred to a nitrocellulose membrane. The membranes are preferably incubated with rabbit polyclonal anti-phospho-GSK-3α/β (Ser21/9) antibody (available from Cell Signaling Technology, Beverly, Mass., catalog # 9331).

Competition experiments with ATP. Concentrations of a test compound (for example, phenoxazine compound 15B; a specific phenoxazine of formula (I), infra) can be prepared as 10× stocks in DMSO ranging from 25 μM to 50 mM, to give a final reaction concentration range of 2.5 μM to 5 mM. An ATP master mix can also be prepared containing 0.75 μl [γ³³P] ATP (available from Perkin-Elmer, Boston, Mass., catalog number NEG302H), 0.5 μl of 10 mM ATP, and 1.25 μl of 1× kinase buffer (20 mM MOPS, pH 7.2; 25 mM β-glycerol phosphate; 5 mM EGTA; 1 mM Na₃VO₄; and 1 mM DTT) for each sample. An enzyme/substrate master mix can be prepared containing 10 μl of the 1× kinase buffer, 5 μl of AKT peptide substrate stock (available from Upstate Biotechnology, Lake Placid, N.Y.) diluted to 670 ng/μl using the 1× kinase buffer, and 5 μl of active AKT (10 ng/μl) (also available from Upstate Biotechnology) diluted from stock using the 1× kinase buffer. The reactions can be set up by adding 2.5 μl of the test compound to the bottom of the tube followed by the addition of 2.5 μl of ATP mix near the bottom of the tube. The reaction can be initiated by the addition of 20 μl of the enzyme/substrate master mix. After adding the master mix to all of the tubes, the samples are incubated at 30° C. for 30 minutes. The sample can be then centrifuged briefly and spotted onto phosphocellulose squares in the same order as the addition of the master mix. These samples can then be added to a beaker with 0.75% phosphoric acid, preferably after two minutes and in the same order as above. The samples are then washed for five minutes in 0.75% phosphoric acid three times, followed by five minutes in acetone. The squares are then placed in Whatman paper and allowed to dry. Radioactivity can be quantitated by scintillation counting.

PI 3-kinase assay. 20 ng of recombinant p-110 gamma enzyme (available from AG Scientific, San Diego, Calif.), DMSO (5 μl), test compound (e.g., a phenoxazine or acridone compound, preferably 5 μM), or wortmannin (5 μM) are preferably placed on ice for 1 hour in 100 μl of 1× kinase buffer (10 mM Tris, pH 7.4; 100 mM NaCl; and 5 mM MgCl₂). 10 μg of phosphatidylinositol (available from Sigma, St. Louis, Mo.) can then be added to each sample, and the incubation preferably continues on ice for an additional 15 minutes. ATP (final concentration 25 μM containing 30 μCi of [γ³²p]-ATP) can be added to each sample, and the reaction mixtures incubated at 37° C. for 10 minutes. Reactions can be terminated by adding 20 μl of 6 N hydrochloric acid. The sample is preferably vortexed, and lipids extracted into 300 μl of MeOH:CHCl₃ (1:1) mixture. After mixing gently and spinning at 10,000×g for 5 minutes, 50 μl of the organic phase is preferably spotted onto a silica coated thin layer chromatography (TLC) plate (available from EMD, La Jolla, Calif.) and developed using a solvent system containing CHCl₃:MeOH:H₂O:NH₄OH (60:47:11.3:2). The TLC plate can then be allowed to dry, and the bands analyzed using a Storm 860 phosphoimager (available from Amersham Biosciences, Sunnyvale, Calif.).

PDK1 and SGK1 kinase assays. In vitro PDK1 activity assays can be performed using a PDK1 assay kit (available from Upstate Biotechnology, Lake Placid, N.Y.), preferably with the following modification of the manufacturer's instructions. Briefly, 10 ng of recombinant PDK1 enzyme and 5 μl of DMSO or of test compound in DMSO (e.g., a phenoxazine or acridone compound, preferably 5 μM) are incubated in 80 μl of 1×PDK-assay dilution buffer (50 mM Tris-HCl pH 7.5, 0.1 mM EGTA, 0.1 mM EDTA, 0.1% (v/v) 2-mercaptoethanol, 2.5 μM PKI, 1 μM Microcystin-LR, 10 mM magnesium acetate, and 0.1 mM ATP) on ice. After 1 hour, 100 ng of serum glucocorticoid regulated kinase 1 (SGK1) is added to each sample and incubated on ice for an additional ten minutes. The samples are transferred to a 30° C. water-bath and incubated for an additional 15 minutes. Then, 245 μM of SGK1 substrate peptide (Upstate Biotechnology, Lake Placid, N.Y., catalog # 12-340) followed by ATP (40 μM containing 10 μCi of [γ³²p]-ATP) are added and the reaction mixture is gently vortexed. Samples are incubated at 30° C. for 15 minutes with a gentle vortexing every 2 minutes. Samples are centrifuged, and 40 μl of the reaction mixture is spotted onto the center of a PE 81 phosphocellulose paper square (Upstate Biotechnology catalog number 20-134). After 30 seconds, the filter is washed 4 times with 0.75% phosphoric acid, and twice with acetone. The filter is then drained and transferred into a scintillation vial to which 5 ml of scintillation cocktail is added. The amount of incorporated radioactivity into the substrate can be determined by routine scintillation counting. The assay of SGK1 kinase activity is performed as described above for the PDK1 assay. To test for inhibition of SGK1 by a test compound (e.g., one of the phenoxazine compounds described infra) the SGK1 is incubated on ice with the test compound for one hour prior to addition of activated PDK1.

Translocation of AKT in Rh1 cells. Rh1 cells (2×10⁵ per chamber) can be grown on 2-well glass chamber slides (available from Falcon, Franklin Lakes, N.J.) in serum-free medium containing fibronectin (10 μg/ml). Preferably after twenty hours, the cells are exposed to DMSO (0.1%, vehicle control) or test compound (e.g., 5 μM of phenoxazine or acridone compound) for one hour and then stimulated with IGF-I (10 ng/ml) for 20 minutes. Cells are preferably washed twice with PBS and fixed in 4% formaldehyde for 30 minutes at room temperature. The samples are then rinsed twice with PBS and permeabilized with 1% Triton X-100 for five minutes at room temperature. After rinsing twice with PBS, the cells are incubated with an anti-AKT antibody (available from Rockland, West Chester, Pa.) (1:50 dilution) for 45 minutes at 37° C. After rinsing three times with PBS, the slides are then incubated with an anti-IgG rabbit secondary antibody coupled to Alexa 488 (available from Molecular Probes, Eugene, Oreg.) at a dilution of 1:50. The slides are preferably washed and incubated with RNase. After rinsing twice with PBS, the slides can be mounted in media containing TOPRO-3 (also available from Molecular Probes) and analyzed by routine confocal microscopy.

Cell growth inhibition. Rh1, Rh18 and Rh30 cells at a density of 6,000; 50,000 and 10,000 cells, respectively, are plated per well in 6-well flat bottom tissue culture plates (available from Falcon, Franklin Lakes, N.J.) in complete medium. After 24 hours at 37° C., the culture medium is replaced with fresh medium containing DMSO (0.1%) or with test compound (e.g., a phenoxazine or acridone compound) at concentrations ranging from 100 nM to 25 μM. The cells are further incubated for six days. Growth can be assessed after lysing cells, and counting nuclei. All measurements are preferably made in triplicate.

Determination of apoptosis. An ApoAlert™ Annexin V-FITC Apoptosis kit (available from Clontech, Palo Alto, Calif.) can be used to evaluate the extent of apoptosis within cell populations. Cells (Rh1: 350,000 per 75-cm² flask; Rh18: 800,000 per 75-cm² flask; or Rh30: 500,000 per 75-cm² flask) are preferably grown overnight in complete medium. On day 1, cells are treated with DMSO (0.1%; vehicle control) or with a test compound (e.g., a phenoxazine or acridone compound). After 4 days, the cells are trypsinized, washed with PBS, and resuspended in 200 μl of binding buffer. Cells are then incubated with 10 μl of annexin V-FITC (final concentration, 1 μg/ml) and 500 ng of propidium iodide in a final volume of 410 μl. Cells are preferably incubated at room temperature in the dark for ten minutes before flow cytometric analysis with an FACSCalibur™ Flow Cytometry System (Becton Dickinson, San Jose, Calif.).

7.2. Inhibition of AKT Phosphorylation in Cells by Phenoxazine Compounds

Several phenoxazine compounds for Formula (I), below, were investigated to determine the ability to inhibit AKT phosphorylation.

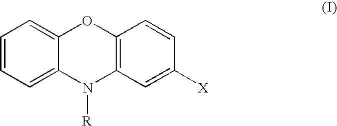

In particular, Table I below list several exemplary phenoxazine compounds that were assayed according to the experimental protocols of these examples. The table also provides the identity of each functional group, —R and —X from formula (I) above, for each of the assayed compounds.

TABLE I
EXEMPLARY PHENOXAXINE DERIVATIVES OF FORMULA (I)

| Compound | | | | |
|---|---|---|---|---|
| ID* | X | R | Name | Inhibition** |
| 1B | Cl | —H | 2-chlorophenoxazine | + |
| 2B | Cl | —(CH₂)₃—Cl | 10-(3′-chloropropyl)-2- | −−− |
| | | | chlorophenoxazine | |
| 3B | Cl | —(CH₂)₃—N(CH₂CH₃)₂ | 10-[3′-(N-diethylamino)- | ++ |
| | | | propyl]-2-chlorophenoxazine | |
| 4B | Cl | —(CH₂)₃—N(CH₂CH₂OH)₂ | 10-[3′-[N-bis(hydroxyethyl) | ++ |
| | | | amino]propyl]-2- | |
| | | | chlorophenoxazine | |
| 5B | Cl | 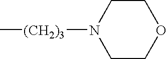 | 10-(3′-N-morpholinopropyl)- 2-chlorophenoxazine | −−− |
| 6B | Cl | 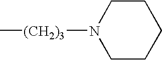 | 10-(3′-N-piperidinopropyl)-2- chlorophenoxazine | ++ |
| 7B | Cl | 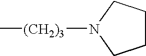 | 10-(3′-N-pyrrolidinopropyl)-2- chlorophenoxazine | +++ |
| 8B | Cl | 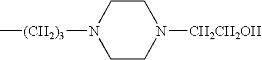 | 10-[3′-[(β-hydroxyethyl) piperazino]propyl]-2- chlorophenoxazine | +++ |
| 9B | Cl | —(CH₂)₄—Cl | 10-(4′-chlorobutyl)-2- | −−− |
| | | | chlorophenoxazine | |
| 10B | Cl | —(CH₂)₄—N(CH₂CH₃)₂ | 10-[4′-(N-diethylamino)butyl[ | ++++ |
| | | | -2-chlorophenoxazine | |
| 11B | Cl | —(CH₂)₄—N(CH₂CH₂OH)₂ | 10-[4′-[N-bis(hydroxyethyl) | +++ |
| | | | amino]butyl]-2- | |
| | | | chlorophenoxazine | |
| 12B | Cl | 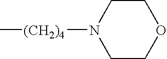 | 10-(4′-N-morpholinobutyl)-2- chlorophenoxazine | −−− |
| 13B | Cl | 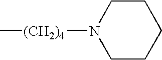 | 10-(4′-N-piperidinobutyl)-2- chlorophenoxazine | +++ |
| 14B | Cl | 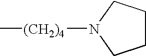 | 10-(4′-N-pyrrolidinobutyl)-2- chlorophenoxazine | +++ |
| 15B | Cl | 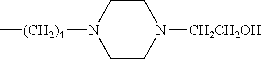 | 10-[4′-[(β-hydroxyethyl) piperazino]butyl]-2- chlorophenoxazine | ++++ |
| 16B | Cl | —COCH₂Cl | 10-(chloroacetyl)-2- | −−− |
| | | | chlorophenoxazine | |
| 17B | Cl | —COCH₂—N(CH₂CH₃)₂ | 10-[(N-diethylamino)acetyl]- | −−− |
| | | | 2-chlorophenoxazine | |
| 18B | Cl | 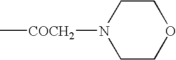 | 10-(N-morpholinoacetyl)-- 2-chlorophenoxazine | −−− |
| 19B | Cl | 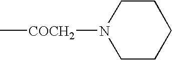 | 10-(N-piperidinoacetyl)-- 2-chlorophenoxazine | −−− |
| 20B | Cl | 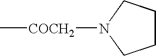 | 10-(N-pyrrolidinoacetyl)-2- chlorophenoxazine | −−− |
| 21B | Cl | 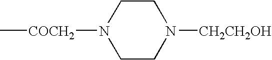 | 10-[[(β-hydroxyethyl) piperazino]acetyl]-2- chlorophenoxazine | −−− |
| 5C | CF₃ | 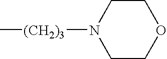 | 10-(3′-N-morpholinopropyl)-- 2-trifluoromethylphenoxazine | −−− |
| 11C | CF₃ | —(CH₂)₄—N(CH₂CH₂OH)₂ | 10-[4′-[N-bis(hydroxyethyl) | +++ |
| | | | amino]butyl]-2- | |
| | | | trifluoromethyl phenoxazine | |
| 13C | CF₃ | 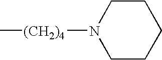 | 10-(4′-N-piperidinobutyl)-- 2-trifluoromethylphenoxazine | +++ |
| 4A | H | —(CH₂)₃—N(CH₂CH₂OH)₂ | 10-[3′-[N-bis(hydroxyethyl) | ++ |
| | | | amino]propyl]phenoxazine | |
| 8A | H | 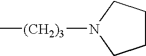 | 10-(3′-N-pyrrolidinopropyl)- phenoxazine | ++ |
| 11A | H | —(CH₂)₄—N(CH₂CH₂OH)₂ | 10-[4′-[N-bis(hydroxyethyl) | +++ |
| | | | amino]-butyl]phenoxazine | |
| 14A | H | 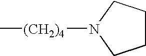 | 10-(4′-N-pyrrolidinobutyl)- phenoxazine | +++ |
| 15A | H | 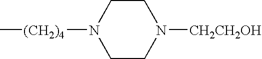 | 10-[4′-[(β-hydroxyethyl piperazino]butyl]-phenoxazine | +++ |
| 22A | H | 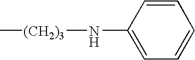 | 10-(3′-N-benzylaminopropyl)- phenoxazine | +++ |

* All at 5 μm concentration
** + ≈ 25% inhibition, ++ ≈ 50% inhibition, +++ ≈ 75% inhibition, ++++ ≈ 100% inhibition (average of two experiments)
−−− <25% inhibition

For these assays, Rh1 cells are seeded in serum-free medium for overnight attachment. The serum starved Rh1 cells are then exposed to 1-5 μM of a compound in Table I for 1 hour before stimulating with IGF-I (10 ng/ml) for 10 minutes. AKT and/or ERK-1/2 phosphorylation can be detected, e.g. by Western blot analysis of cell lysates, using the phospho-specific anti-AKT antibody or anti-ERK-1/2 antibody. IGF-I stimulates phosphorylation of AKT (Ser 473) and ERK-1/2 (Thr202/Tyr204), but has no effect on the overall protein levels of AKT or ERK-1/2.

The results of such analysis shows that, with the possible exception of compounds 5C, 2B, 5B, 9B, 12B and 16B-21B, all of the compounds inhibit phosphorylation of AKT at Ser 473 to at least some degree at a concentration of 5 μM. These results are summarized in the far right-hand column of Table I, above.

None of the phenoxazine compounds inhibits IGF-I stimulated phosphorylation of ERK-1/2. Hence, the phenoxazine compounds are not inhibiting the IGF-I receptor, insulin receptor substrate (IRS) proteins or PI 3-kinase as these pathways are necessary for IGF-I mediated activation of ERK-1/2. The results of such experiments show that for the compounds in Table I, supra, the potency of AKT inhibition follows in the order: n=4 (butyl)>n=3 (propyl) series. Morpholino- and -acetyl derivatives of phenoxazine, in particular, compounds 8A, 4A, 11A, 14A, 15A, 22A, 5C, 11C and 13C, exhibit minimal inhibition of cellular AKT activation at the concentrations examined in this assay.

To determine the minimum concentration at which AKT phosphorylation is inhibited, cells can be grown under serum free conditions and then exposed to compounds, e.g., in Table I at concentrations of 1, 2.5 or 3.5 μM. Phospho-AKT can then be detected after stimulating with IGF-I, as described above. Results from such experiments reveal that exposure to 1 μM concentrations causes about 60% inhibition, whereas exposure to 3.5 μM causes maximum inhibition for most of the compounds in Table I. However, compounds 10B and 15B from Table I are particularly active, and show complete inhibition in these assays at concentrations of 2.5 μM.

7.3. Inhibition of AKT Activation Prevents Activation of Its Downstream Targets

mTOR, p70S6 kinase and rpS6 are downstream targets of AKT signaling (see, e.g., Jacinto et al. Nature Rev. Mol. Cell Biol. 2003;4:117-126 and Abraham Cell 2002;111:9-12). Hence, the role of AKT activity in the generation of phospho-mTOR (mTOR phosphorylated on the AKT dependent phosphorylation site Ser2448 and/or the autophosphorylation site Ser2481), phospho-p70S6 kinase (Thr389), or phospho-rpS6 (Ser235/236) can be assessed, e.g. by Western blot analysis of cell lystates, by pretreating Rh1 cells grown in serum-free medium with test compounds (e.g. phenoxazine compounds 3B, 8B, 10B, 12B or 15B) for one hour at concentrations of 3.5 to 5.0 μM, followed by stimulation with IGF-I for 10 minutes. The results of such experiments show that the IGF-I induced phosphorylation of mTOR (Ser2448 and Ser2481), rpS6 (Ser235/236) and p70S6 kinase (Thr389) are markedly inhibited by the compounds 8B, 10B and 15B and, to a lesser extent, by compound 12B.

Hence, results from such experiments show that phenoxazine compounds such as those listed in Table I, above, have the ability to shut down the survival AKT/mTOR pathway in Rh1 cells.

These experiments can also be performed using other cell lines, e.g. serum-starved Rh18 and Rh30 rhabdomyosarcoma cells. In both cell lines, IGF-1-induced phosphorylation of AKT, mTOR and rpS6 is effectively blocked by all of the compounds, with the possible exception of compound 20B.

To confirm that equal amounts of protein are loaded in such experiments, the membrane can be stripped of bound antibodies, and incubated with the anti-AKT antibody to determine the total amount of AKT protein.

Such experiments demonstrate that AKT mediated activation of mTOR/p70S6 kinase/rpS6 pathways in various cancer cell lines can be blocked by phenoxazine compounds such as those identified in Table I, above.

7.4. Phenoxazine compounds Inhibit AKT Kinase Activity in Cells

To determine directly whether compounds, such as the phenoxazine compounds in Table I, inhibit AKT activation in cells, the activation of AKT by IGF-I can be evaluated by assessing either phosphorylation of AKT (Ser473), or the in vitro kinase activity of protein immunoprecipitated by anti-AKT antibody. In particular, the phosphorylation status of a downstream target of AKT, e.g., GSK-3β, can be examined to determine whether changes in AKT phosphorylation correlate with alterations in AKT kinase activity. For example, Rh1 cells grown in serum-free medium can be exposed to 0.1% DMSO or 5 [M of test compound (e.g., compound 10B or 15B from Table I) for one hour and then stimulated with IGF-I for 10 minutes. Cell lysates can then be immunoprecipitated with immobilized anti-AKT antibody, and the immunoprecipitates used in vitro to phosphorylate a GSK-3 fusion protein (Cell Signaling Technology, Beverly, Mass., catalog #9278). The results of such experiments show that phosphorylation of a GSK-3 fusion protein is completely inhibited in cells treated with these compounds, demonstrating that phenoxazines effectively block the activity of endogenous AKT in cells.

7.5. Phenoxazines Do Not Inhibit PI 3-Kinase Activity In Vitro

As explained above, the finding that phenoxazine compounds do not inhibit IGF-I induced phosphorylation of ERK-1/2 shows that they do not inhibit PI 3-kinase. However, cells treated with phenoxazines do exhibit many of the effects observed in cells treated with PI 3-kinase inhibitors such as wortmannin. This phenomenon can be explained by the fact that PI 3-kinase is required both for association of AKT with the cell membrane by the pleckstrin homology (PH) domain of AKT, and for activation of the AKT kinase function through phosphorylation of Ser308 by the 3-phosphoinositide-dependent protein kinase PDK1.

In vitro kinase assays can be performed using recombinant p-110 gamma enzyme to verify that the phenoxazine and acridone compounds of the present invention do not target PI 3-kinase. For example, kinase activity can be compared between an untreated sample, sample treated with a known PI 3-kinase inhibitor (e.g., wortmannin), and sample(s) treated with 5 μM of test compound(s) (e.g. any of the phenoxazine compounds in Table I) using phosphatidylinositol (PI) as a substrate and [γ³²P]-ATP as the phosphate donor. Lipids in such assays can be resolved by thin layer chromatography (TLC), and incorporated radiolabel quantitated using a phosphoimager. In such experiments, the untreated sample (i.e., sample treated only with DMSO control) shows robust phosphorylation of PI, as indicated by the levels of phosphatidylinositol 3 phosphate (PI(3)P) detected. PI 3-kinase activity in samples treated with 5 μM of test compound 10B or 15B is comparable to the untreated sample, whereas the wortmannin treated sample has barely detectable levels of PI 3-kinase activity, if any. The results from such assays therefore demonstrate that phenoxazine compounds and other compounds, such as those in Table I above, do not inhibit the activity of PI 3-kinase.

7.6. Phenoxazines Do Not Inhibit SGK1 or PDK1 Kinase Activity

The AKT proteins represent a subfamily of the AGC family of kinases. Assays can also be performed to determine whether a test compound (e.g., a phenoxazine compound such as those listed in Table I, above) is capable of modulating the activity of another AGC family member besides AKT and, in particular, to evaluate whether modulation of another AGC family member's activity might contribute to observed effects in assays (for example, the assays described above) using AKT.

For example, an in vitro coupled-kinase assay can be performed using recombinant SGK1, an AGC family member that is closely related to AKT. Recombinant, inactive SGK1 can be pre-incubated for one hour with a test compound (e.g., a phenoxazine compound such as 10B, 15B or another compound from Table I) or with DMSO as a negative control. The pre-incubated SGK1 is then incubated with recombinant, pre-activated PDK1 and ATP for 15 minutes at 30° C., resulting in the activation of SGK1 by phosphorylation (Thr256). Substrate peptide (Upstate Biotechnology, Lake Placid, N.Y., catalog # 12-340) is added to the activated SGK1 reaction mixture together with [γ³²P]-ATP. The reaction is allowed to proceed for some fixed time (e.g., fifteen minutes), and the radiolabel incorporated in the peptide quantitated, e.g., by binding to a phosphocellulose filter and scintillation counting. Because PDK1 is also a member of the AGC family, it is preferable to also perform experiments investigating the possibility that the test compound might interfere with the SGK1 assay by modulating PDK1 activity. This can be done in a control experiment where PDK1 is pre-incubated with the test compound(s) prior to activation and addition to SGK1.

The results from such an assay show that other AGC family members such as SGK1 and PDK1 are not affected by the compounds of this invention.

7.7. Phenoxazines Inhibit AKT Kinase Activity in an In Vitro Assay

The phosphorylation status of GSK-3 protein can also be used to study the AKT inhibitory activity of phenoxazines (including the phenoxazine compounds listed in Table I above) and acridone compounds. For example, recombinant AKT1 or recombinant AKT lacking the pleckstrin homology domain (e.g., expressed in Sf21 cells, 10 ng/reaction) can be pre-incubated with a test compound (e.g., one of the phenoxazine compounds listed in Table I, such as 10B or 15B) at 5 μM for two hours on ice prior to initiation of a kinase assay as described in Section 7.1, above. The results of such experiments show that phosphorylation of GSK-3 is completely blocked by compound 15B, and that inhibition of GSK-3 phosphorylation by compound 10B is at least nearly complete. Hence, such experiments demonstrate that test compounds, including phenoxazine compounds such as those listed in Table I, directly target and inhibit the kinase function of AKT.

7.8. Phenoxazines Do Not Block AKT Activation By the Pleckstrin Homology Domain

All AKT isoforms have a conserved domain structure that includes: an amino terminal pleckstrin homology (PH) domain, a central kinase domain, and a carboxyl-terminal regulatory domain that contains the hydrophobic motif, a characteristic of AGC family kinases. The PH domain is a phosphoinositide-binding motif found in a number of signal-transducing proteins, including but not limited to AKT proteins, the gives the protein membrane-binding properties. In particular, the PH domain interacts with membrane lipid products such as phosphatidylinositol(3,4,5)trisphosphate (PtdIns(3,4,5)P3] produced by PI 3-kinase (See, e.g., Frech et al. J. Biol. Chem. 1997;272:8474-8481). Biochemical analysis has revealed that the PH domain of AKT binds to both PIP3 and PIP2 with similar affinity (James et al. J. Biochem. 1996;315:709-713 and Vazquez et al. Biochim. Biophys. Acta. 2000; 1470:M21-M35), recruiting AKT to the plasma membrane (Cantley et al. Proc. Natl. Acad. Sci. USA 1999;96:4240-4245; Vazquez et al. Biochim. Biophys. Acta. 2000;1470:M21-M35; and Leevers et al. Curr. Opin. Cell Biol. 1999;11:219-225). PIP2 binding to the PH domain induces a conformation change in AKT, exposing a critical Thr308 residue in the activation loop to phosphorylation by PDK1. For full activation, AKT is subsequently phosphorylated at Ser473 by an as yet unidentified kinase referred to as phosphoinositide 3 phosphate dependent kinase 2 (PDK2) (See, e.g., Cantley et al. Proc. Natl. Acad. Sci. USA 1999;96;4240-4245; Vazquez et al. Biochim. Biophys. Acta. 2000;1470:M21-M35; and Coffer et al. J. Biochem. 1998;335:1-13).

The observation that a test compound does not inhibit PDK1 activity (e.g., in experiments such as those described above) may indicate that interaction with the PH domain of AKT is not necessary for the inhibitory effects of a test compound. It is therefore preferable to determine, in such instances, whether the absence of a PH domain in AKT can affect the ability of a test compound (for example, a phenoxazine compound such as 10B, 15B or another compound from Table I) to inhibit AKT kinase activity. For example, in vitro kinase assays can be performed using a recombinant AKT isoform, referred to herein as AKTΔPH, that lacks the PH domain. Since GSK-3 is a downstream phosphorylation target of AKT, its phosphorylation can be used as an indication of AKT activity in such an assay.

The results of such experiments show that deletion of the PH domain results in a higher level of kinase activity than the full-length AKT. However, the ability of both compounds 10B and 15B to inhibit AKT kinase activity is unaffected by deletion of the PH domain.

Results from such experiments demonstrate that compounds of the invention, including phenoxazine compounds such as those listed in Table I above, do not mediate their effects by interacting with the PH domain of AKT, or by blocking the association of AKT with the cell membrane.

7.9. Phenoxazines Block Translocation of AKT from the Cytoplasm to the Nucleus

Upon activation, AKT translocates to the nucleus (see, e.g., Biggs et al. Proc. Natl. Acad. Sci. USA 1999;96:7421-7426; Brownawell et al. Mol. Cell. Biol. 2001;21:3534-3546; Brunet et al. Cell 1999;96:857-868; and Rena et al. J. Biol. Chem. 1999;274:17179-17183). Hence, a predicted effect of inhibiting AKT with a compound of this invention is a decrease in localization to the nucleus in response to growth factor stimulation. This can be investigated in confocal microscopy experiments using an anti-AKT antibody to examine cellular localization of AKT protein in response to treatment with a test compound (for example, with a Phenoxazine compound such as 10B, 15B or another compound listed in Table I). For example, Rh1 cells can be placed in chamber well slides in MN2E medium for 20 hours, followed by the addition of 5 μM of test compound or DMSO (0.1%) vehicle control for one hour, after which time 10 ng/ml of IGF-1 is added for 20 minutes. The cells are then fixed and incubated with anti-AKT antibody as well as with the DNA-intercalating fluorescent dye TOPRO-3 (Molecular Probes, Eugene, Oreg.) to identify the nucleus. Cellular localization of AKT may then be assessed, e.g. by confocal microscopy.

Results from such experiments demonstrate that a block in nuclear localization occurs when AKT activation is inhibited using compounds of the invention, including phenoxazine compounds such as 10B, 15B and other compounds listed in Table I.

7.10. Phenoxazines Inhibit Cell Growth

The effect(s) of compounds, including phenoxazine compounds such as those listed in Table I, above, on cell growth can be evaluated in cell-based assays such as the exemplary assays described here. Rh1, Rh18 and/or Rh30 cells grown in complete medium can be exposed to graded concentrations of test compound (e.g., from 0.1 to 25 μM) for six days, at which time the cells can be lysed and their growth assessed by counting nuclei. Using such cell counts, graphs depicting the typical effect of graded concentrations of test compounds (e.g., phenoxazine compounds 10B, 15B, 12B, and 20B) on the growth of Rh1 cells may be plotted.

Results from such experiments show that all three cell lines (Rh1, Rh18 and Rh30) are sensitive to both the compounds 10B and 15B, with typical IC₅₀ values of 2 μM, 5 μM and 6 μM for the Rh1, Rh18 and Rh30 cells respectively. These levels of growth inhibition correlate well with the concentration of the compounds that inhibit AKT in cell-based assays. In contrast, the compounds 12B and 20B are about 10-fold or more less inhibitory in such cell growth assays. This is consistent with the relative lack of AKT inhibition observed for these compounds in comparable cell based assays.

7.11. Phenoxazines Induce Apoptosis

The effect(s) of compounds on cell apoptosis can also be investigated in exemplary assays that are described here. For instance, Rh1, Rh18 and/or Rh30 cells can be grown in complete medium with 0.1% DMSO (as a negative control) or with one or more test compounds, e.g., any of the phenoxazine compounds listed in Table I, above, including but not limited to the compounds 10B, 11B, 13B, 14B or 15B. In particular examples described and demonstrated here, the cells are incubated with the test compound(s) at concentrations of 6.5 μM (in Rh1 cells) or 7.5 μM (in Rh18 and/or Rh30 cells) for four days. Cells are then harvested, and the extent of apoptosis evaluated, e.g., by an ApoAlert™ (Clontech, Palo Alto, Calif.) flow cytometric assay.

Within apoptotic cells populations, cells in the early stages of apoptosis are annexin V-positive and propidium iodide negative, whereas cells in the late stages of apoptosis are both annexin V-positive and propidium iodide negative. Exemplary data from combined populations of cells are presented in Table II, below.

TABLE II
PHENOXAXINE INDUCED APOPTOSIS
IN RHABDOMYOSARCOMA CELLS

| | |
|---|---|
| Cell line + | Percentage of cells ± SD^{a} |

| treatment^{b} | Viable | Apoptotic^{c} |
|---|---|---|
| Rh1 control | 82.33 ± 5.44 | 17.70 ± 5.43 |
| Rh1 + 10B | 47.70 ± 7.90 | 52.00 ± 8.29 |
| Rh1 + 15B | 23.33 ± 8.66^{d} | 75.33 ± 8.50 |
| Rh18 control | 79.70 ± 5.31 | 19.33 ± 5.31 |
| Rh18 + 10B | 65.00 ± 2.94 | 34.33 ± 6.13 |
| Rh18 + 15B | 67.00 + 7.48 | 32.70 ± 8.99 |
| Rh30 control | 89.67 ± 1.89 | 10.00 ± 2.16 |
| Rh30 + 10B | 56.67 ± 2.62 | 43.00 ± 2.17 |
| Rh30 + 15B | 11.00 + 2.16^{d} | 88.67 ± 1.69 |

^{a}Results are mean ± SD (n = 3).
^{b}6.5 μM of 10B or 15B for Rh1; 7.5 μM of 10B or 15B for Rh18 and Rh30.
^{c}Necrosis, Annexin V-negative, propidium iodide-positive: <1.5%.
^{d}P < 0.05

Approximately 10 to 19% of cells in control populations (i.e., cells exposed only to DMSO) undergo spontaneous apoptosis. Treatment with compound 10B in Table I results in about 52% apoptosis in Rh1 cells, 34% apoptosis in Rh18 cells, and 43% apoptosis in Rh30 cells. Treatment of Rh1, Rh18 and Rh30 cells with compound 15B in Table I results in about 75%, 33% and 89% apoptosis, respectively. A significant increase in the proportion of apoptotic cells is also evident after treatment with other compounds of the invention, including the compounds 11B, 13B and 14B from Table I.

Similar experiments can be performed using compounds that are relatively poor inhibitors of AKT in vitro but, preferably, are chemically similar to the phenoxazine or other compounds tested that are effective inhibitors of AKT. For example, the apoptosis of cells in response to the phenoxazine compound 12B or 20B, which are relatively poor inhibitors of AKT in vitro, can be compared to apoptosis of cells in response to the chemically similar compounds 10B and/or 15B, which are effective AKT inhibitors. In this way, a skilled practitioner can evaluate whether apoptosis observed in response to an effective AKT inhibitor (e.g., apoptosis observed in response to compound 10B or 15B) is due to a general toxic effect rather than AKT inhibition. In contrast to the effect of AKT inhibitor compounds such as 10B and 15B, neither the compound 12B or 20B (both of which are relatively poor AKT inhibitors in vitro) induces apoptosis.

Data from such experiments establish that phenoxazine and other compounds of this invention (including compounds listed in Table I, above) effectively induce apoptosis in cells and, moreover, that there is a correlation between this effect and the compounds' ability to inhibit AKT.

7.12. Effect of Acridone compounds on AKT Phosphorylation in Cells

Several acridone compounds having the chemical formula of formula (III), below, can also be screened, e.g., in any of the assays described above, to investigate their ability to inhibit AKT activity and, in particular, to inhibit phosphorylation of AKT at Ser473 in cells.

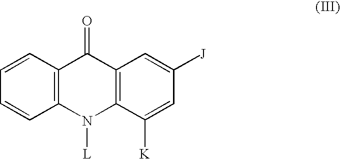

Examples of some preferred acridone compounds that can be screened in such assays and/or used in accordance with the invention, including the compounds listed in Table III, below.

TABLE III
EXEMPLARY ACRIDONE COMPOUNDS OF FORMULA (III)

| Compound | |
|---|---|
| ID | Name |
| 1 | 10-(3′-N-Diethylaminopropyl)-2-chloroacridone |
| 2 | 10-[3′-N-(Methylpiperazino)propyl]-2-chloroacridone |
| 3 | 10-(3′-N-Piperidinopropyl)-2-chloroacridone |
| 4 | 10-[3′-N-Pyrrolidinopropyl]-2-chloroacridone |
| 5 | 10-(3′-N-Morpholinopropyl)-2-chloroacridone |
| 6 | 10-(3′-Chloropropyl)-2-chloroacridone |
| 7 | 10-(4′-N-Diethylaminobutyl)-2-chloroacridone |
| 8 | 10-(4′-N-(Methylpiperazino)butyl)-2-chloroacridone |
| 9 | 10-(4′-N-Piperidinobutyl)-2-chloroacridone |
| 10 | 10-(4′-N-[(β-Hydroxyethyl)piperazino]butyl)-2- |
| | chloroacridone |
| 11 | 10-[4′-N-Pyrrolidinobutyl]-2-chloroacridone |
| 12 | 10-(4′-N-Morpholinobutyl)-2-chloroacridone |
| 13 | 10-(4′-Chlorobutyl)-2-chloroacridone |
| 14 | 10-(4′-N-Piperidinobutyl)-2-methoxyacridone |
| 15 | 10-(4′-N-([β-Hydroxyethyl]piperazino)butyl)-2- |
| | bromoacridone |
| 16 | 10-(3′-N-[(β-Hydroxyethyl) piperazino] propyl)-2- |
| | bromoacridone |
| 17 | 10-(3′-N-[Bis[hydroxyethyl]amino]propyl)-2- |
| | bromoacridone |
| 18 | 10-(4′-N-Chlorobutyl)-2-bromoacridone |
| 19 | 10-(3′-N-Morpholinopropyl)-2-bromoacridone |
| 20 | 10-(4′-[N-Diethylamino)butyl)-2-bromoacridone |
| 21 | 10-(4′-N-Pyrrolidinobutyl)-2-bromoacridone |
| 22 | 10-(4′-N-Morpholinobutyl)-2-bromoacridone |
| 23 | 10-(3′-N-Piperidinopropyl)-2-bromoacridone |
| 24 | 10-(4′-N-Thiomorpholinobutyl)-2-bromoacridone |
| 25 | 10-(3′-N-Pyrrolidinopropyl)-2-bromoacridone |
| 26 | 10-(3′-[N-Diethylamino]propyl)-2-bromoacridone |

For example, Rh1 cells can bee seeded in MN2E medium for overnight attachment, and then exposed to an acridone compound of formula (III) at 1, 5 or 10 μM concentration. After exposing the cells to a test compound for a particular amount of time (preferably for one hour), the cells can be stimulated with IGF-I (10 ng/ml) for ten minutes. The cell lysates are then resolved by SDS-PAGE and immunoblotted for phospho-AKT (Ser473), as described above.

The results from such experiments show that acridone compounds and, in particular, compounds 2, 6-10, 13, 21, 22, 25 and 26 from Table III, above, effectively inhibit the phosphorylation of AKT in Rh1 cells at concentrations <5 μM.

### 8. REFERENCES CITED

Numerous references, including patents, patent applications and various publications, are cited and discussed in the description of this invention. The citation and/or discussion of such references is provided merely to clarify the description of the present invention and is not an admission that any such reference is “prior art” to the invention described here. All references cited and/or discussed in this specification (including references, e.g., to biological sequences or structures in the GenBank, PDB or other public databases) are incorporated herein by reference in their entirety and to the same extent as if each reference was individually incorporated by reference.

What is claimed is:

1. A method of inhibiting cell growth of a cell, said method comprising contacting the cell with an effective amount of a phenoxazine compound, or a pharmaceutically acceptable salt thereof, wherein the phenoxazine compound is of Formula (I): 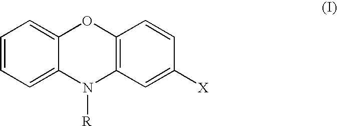
wherein
X is haloalkyl; and
R is selected from hydrogen and (CH₂)ₙA;
wherein
n is an integer selected from 2, 3, 4, 5, and 6; and
A is selected from —NR₁R₂;
wherein
R₁ and R₂ are independently selected from hydrogen, linear or branched alkyl, linear or branched alkyl substituted with one or more hydroxyl groups, phenyl, and substituted phenyl; or
R₁ and R₂ when taken together with the nitrogen atom to which they are attached, optionally form a cyclic ring of the formula (II): 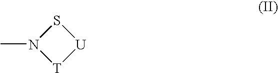
wherein
S and T are independently alkylene having 1, 2, 3, or 4 carbon atoms; and
U is selected from —O—, —S—, —N(R₃)—, and —CH(R₄)—;
wherein
R₃ and R₄ are independently selected from hydrogen, linear or branched alkyl, and linear or branched alkyl substituted with one or more hydroxyl groups.

2. The method of claim 1, wherein
S and T are independently alkylene having 1, 2, 3, or 4 carbon atoms; and
U is selected from —O—, —S—, —N(R₃)—, and —CH(R⁴)—; with the proviso that when S and T are both —(CH₂)₂—, U is not —O—.

3. The method of claim 2, wherein n is 3 or 4.

4. The method of claim 2, wherein R₁ and R₂ are independently selected from ethyl, n-propyl, ω-hydroxyethyl and ω-hydroxypropyl.

5. The method of claim 2, wherein the phenoxazine compound of Formula (I) is selected from:
10-[4′-[N-bis(hydroxyethyl)amino]butyl]-2-trifluoromethyl phenoxazine, and
10-(4′-N-piperidinobutyl)—2-trifluoromethylphenoxazine.
and pharmaceutically acceptable salts thereof.

6. A method of treating cancer in a patient, said method comprising administering to a patient in need of such treatment an effective amount of a phenoxazine compound, or a pharmaceutically acceptable salt thereof, wherein the phenoxazine compound is of Formula (I): 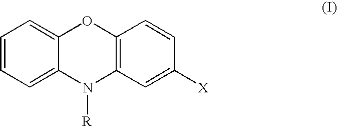
wherein
X is haloalkyl; and
R is selected from hydrogen and (CH₂)ₙA;
wherein
n is an integer selected from 2, 3, 4, 5, and 6; and
A is selected from —NR₁R₂;
wherein
R₁ and R₂ are independently selected from hydrogen, linear or branched alkyl, linear or branched alkyl substituted with one or more hydroxyl groups, phenyl, and substituted phenyl; or
R₁ and R₂ when taken together with the nitrogen atom to which they are attached, optionally form a cyclic ring of the formula (II): 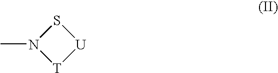
wherein
S and T are independently alkylene having 1, 2, 3, or 4 carbon atoms; and
U is selected from —O—, —S—, —N(R₃)—, and —CH(R₄)—; wherein
R₃ and R₄ are independently selected from hydrogen, linear or branched alkyl, and linear or branched alkyl substituted with one or more hydroxyl groups.

7. The method of claim 6, wherein
S and T are independently alkylene having 1, 2, 3, or 4 carbon atoms; and
U is selected from —O—, —S—, —N(R₃)—, and —CH(R₄)—; with the proviso that when S and T are both —(CH₂)₂—, U is not —O—.

8. The method of claim 7, wherein n is 3 or 4.

9. The method of claim 7, wherein R₁ and R₂ are independently selected from ethyl, n-propyl, ω-hydroxyethyl and ω-hydroxypropyl.

10. The method of claim 6, wherein the phenoxazine compound of Formula (I) is selected from:
10-[4′-[N-bis(hydroxyethyl) amino]butyl]-2-trifluoromethyl phenoxazine, and
10-(4′-N-piperidinobutyl)-2-trifluoromethylphenoxazine.
and pharmaceutically acceptable salts thereof.

11. An acridone compound of Formula (III): 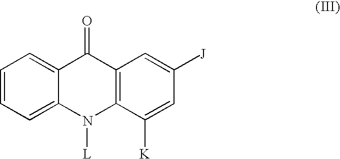
and pharmaceutically acceptable salts thereof,
wherein
J is halogen;
K is selected from hydrogen or alkoxy; and
L is selected from hydrogen and (CH₂)ₙB;
wherein
n is an integer selected from 2, 3, 4, 5, and 6; and
B is selected from halogen and —NR₅R₆;
wherein
R₅ and R₆ are independently selected from hydrogen, linear or branched alkyl, linear or branched alkyl optionally substituted with one or more hydroxyl groups; or
R₅ and R₆ when taken together with the nitrogen atom to which they are attached, optionally form a cyclic ring of the formula (IV): 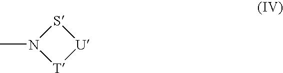
wherein
S′ and T′ are independently alkylene having 1, 2, 3, or 4 carbon atoms; and
U′ is selected from —O—, —S—, —N(R₇)—, and —CH(R₈)—;
wherein
R₇ and R₈ are independently selected from hydrogen, linear or branched alkyl, and linear or branched alkyl substituted with one or more hydroxyl groups.

12. The acridone compound of claim 11, wherein J is selected Cl and Br, and K is selected from hydrogen and OCH₃.

13. The acridone compound of claim 11, wherein the acridone compound of formula (III) is selected from:
10-(3′-N-Diethylaminopropyl)-2-chloroacridone,
10-[3′-N-(Methylpiperazino)propyl]-2-chloroacridone,
10-(3′-N-Piperidinopropyl)-2-chloroacridone,
10-[3′-N-Pyrrolidinopropyl]-2-chloroacridone,
10-(3′-N-Morpholinopropyl)-2-chloroacridone,
10-(3′-Chloropropyl)-2-chloroacridone,
10-(4′-N-Diethylaminobutyl)-2-chloroacridone,
10-(4′-N-(Methylpiperazino) butyl)-2-chloroacridone,
10-(4′-N-Piperidinobutyl)-2-chloroacridone,
10-(4′-N-[(β-Hydroxyethyl)piperazino]butyl)-2-chloroacridone,
10-[4′-N-Pyrrolidinobutyl]-2-chloroacridone,
10-(4′-N-Morpholinobutyl)-2-chloroacridone,
10-(4′-Chlorobutyl)-2-chloroacridone,
10-(4′-N-([β-Hydroxyethyl]piperazino)butyl)-2-bromoacridone,
10-(3′-N-[(β-Hydroxyethyl) piperazino]propyl)-2-bromoacridone,
10-(3′-N-[Bis[hydroxyethyl]amino]propyl)-2-bromoacridone,
10-(4′-N-Chlorobutyl)-2-bromoacridone,
10-(3′-N-Morpholinopropyl)-2-bromoacridone,
10-(4′-[N-Diethylamino)butyl)-2-bromoacridone,
10-(4′-N-Pyrrolidinobutyl)-2-bromoacridone,
10-(4′-N-Morpholinobutyl)-2-bromoacridone,
10-(3′-N-Piperidinopropyl)-2-bromoacridone,
10-(4′-N-Thiomorpholinobutyl)-2-bromoacridone,
10-(3′-N-Pyrrolidinopropyl)-2-bromoacridone, and
10-(3′-[N-Diethylamino]propyl)-2-bromoacridone.
and pharmaceutically acceptable salts thereof.

14. A method of inhibiting cell growth of a cell, said method comprising contacting the cell with an effective amount of the acridone compound of claim 11.

15. A method of treating cancer in a patient, said method comprising administering to a patient in need of such treatment an effective amount of the acridone compound of claim 11.

16. A method of modulating AKT activity, said method comprising contacting an AKT with an effective amount of a phenoxazine compound or an acridone compound, or pharmaceutically acceptable salts thereof.

17. The method of claim 16, wherein contacting an AKT comprises contacting a cell comprising an AKT.

18. A method of inhibiting cell growth of a cell, wherein the cell is a cell in which AKT is activated, said method comprising contacting the cell with an effective amount of a phenoxazine compound or an acridone compound, or pharmaceutically acceptable salts thereof.

19. A method of treating cancer in a patient, wherein the cancer is a cancer in which AKT is activated, said method comprising administering to a patient in need of such treatment an effective amount of a phenoxazine compound or an acridone compound, or pharmaceutically acceptable salts thereof.

20. The method of claim 19, wherein the cancer is gastric cancer, breast cancer, ovarian cancer, pancreatic cancer, prostate cancer, chronic myelogenous leukemia, glioblastoma, endometrial cancer, thyroid cancer, cervical cancer, colorectal cancer, lung cancer, or epithelial carcinoma of the mouth.

21. A method of treating transplant rejection in a patient, said method comprising administering to a patient in need of such treatment an effective amount of a phenoxazine compound or an acridone compound, or pharmaceutically acceptable salts thereof.

22. A method of treating coronary artery disease, said method comprising administering to a patient in need thereof a drug-eluting stent comprising an effective amount of a phenoxazine compound or an acridone compound, or pharmaceutically acceptable salts thereof, wherein the administering comprises placing the drug-eluting stent into the luminal space of at least one coronary artery of the patient.

23. A drug-eluting stent comprising a phenoxazine compound or an acridone compound, or pharmaceutically acceptable salts thereof.

24. The drug-eluting stent of claim 23, wherein the phenoxazine compound is of Formula (I): 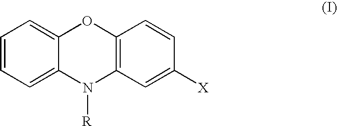
and pharmaceutically acceptable salts thereof,
wherein
X is selected from hydrogen, halogen, and haloalkyl;
R is selected from hydrogen and (CH₂)ₙA;
wherein
n is an integer selected from 2, 3, 4, 5, and 6; and
A is selected from —NR₁R₂;
wherein
R₁ and R₂ are independently selected from hydrogen, linear or branched alkyl, linear or branched alkyl substituted with one or more hydroxyl groups, phenyl, and substituted phenyl; or
R₁ and R₂ when taken together with the nitrogen atom to which they are attached, optionally form a cyclic ring of the formula (II): 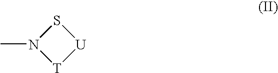
wherein
S and T are independently alkylene having 1, 2, 3, or 4 carbon atoms; and
U is selected from —O—, —S—, —N(R₃)—, and —CH(R₄)—;
wherein
R₃ and R₄ are independently selected from hydrogen, linear or branched alkyl, and linear or branched alkyl substituted with one or more hydroxyl groups.

25. The drug-eluting stent of claim 24, wherein
S and T are independently alkylene having 1, 2, 3, or 4 carbon atoms; and
U is selected from —O—, —S—, —N(R₃)—, and —CH(R₄)—; with the proviso that when S and T are both —(CH₂)₂—, U is not —O—.

26. The drug-eluting stent of claim 25, wherein n is 3 or 4.

27. The drug-eluting stent of claim 25, wherein R₁ and R₂ are independently selected from ethyl, n-propyl, ω-hydroxyethyl and ω-hydroxypropyl.

28. The drug-eluting stent of claim 24, wherein the phenoxazine compound of Formula (I) is selected from:
2-chlorophenoxazine,
10-[3′-(N-diethylamino)-propyl]-2-chlorophenoxazine,
10-[3′-[N-bis(hydroxyethyl) amino]propyl]-2-chlorophenoxazine,
10-(3′-N-piperidinopropyl)-2-chlorophenoxazine,
10-(3′-N-pyrrolidinopropyl)-2-chlorophenoxazine,
10-[3′-[(β-hydroxyethyl) piperazino]propyl]-2-chlorophenoxazine,
10-[4′-(N-diethylamino)butyl]-2-chlorophenoxazine,
10-[4′-[N-bis(hydroxyethyl) amino]butyl]-2-chlorophenoxazine,
10-(4′-N-piperidinobutyl)-2-chlorophenoxazine,
10-(4′-N-pyrrolidinobutyl)-2-chlorophenoxazine,
10-[4′-[(β-hydroxyethyl) piperazino]butyl]-2-chlorophenoxazine,
10-[4′-[N-bis(hydroxyethyl) amino]butyl]-2-trifluoromethyl phenoxazine,
10-(4′-N-piperidinobutyl)-2-trifluoromethylphenoxazine,
10-[3′-[N-bis(hydroxyethyl) amino]propyl]phenoxazine,
10-(3′-N-pyrrolidinopropyl)-phenoxazine,
10-[4′-[N-bis(hydroxyethyl) amino]-butyl]phenoxazine,
10-(4′-N-pyrrolidinobutyl)-phenoxazine,
10-[4′-[(β-hydroxyethyl piperazino]butyl]-phenoxazine, and
10-(3′-N-benzylaminopropyl)-phenoxazine.
and pharmaceutically acceptable salts thereof.

29. The drug-eluting stent of claim 23, wherein the acridone compound is of Formula (III): 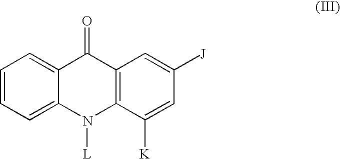
and pharmaceutically acceptable salts thereof,
wherein
J is selected from hydrogen, halogen, or alkoxy;
K is selected from hydrogen or alkoxy; and
L is selected from hydrogen and (CH₂)ₙB;
wherein
n is an integer selected from 2, 3, 4, 5, and 6; and
B is selected from halogen and —NR₅R₆;
wherein
R₅ and R₆ are independently selected from hydrogen, linear or branched alkyl, linear or branched alkyl optionally substituted with one or more hydroxyl groups; or
R₅ and R₆ when taken together with the nitrogen atom to which they are attached, optionally form a cyclic ring of the formula (IV): 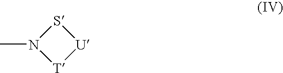
wherein
S′ and T′ are independently alkylene having 1, 2, 3, or 4 carbon atoms; and
U′ is selected from —O—, —S—, —N(R₇)—, and —CH(R₈)—;
wherein
R₇ and R₈ are independently selected from hydrogen, linear or branched alkyl, and linear or branched alkyl substituted with one or more hydroxyl groups.

30. The drug-eluting stent of claim 29, wherein the acridone compound of formula (III) is selected from:
10-(3′-N-Diethylaminopropyl)-2-chloroacridone
10-[3′-N-(Methylpiperazino)propyl]-2-chloroacridone
10-(3′-N-Piperidinopropyl)-2-chloroacridone
10-[3′-N-Pyrrolidinopropyl]-2-chloroacridone
10-(3′-N-Morpholinopropyl)-2-chloroacridone
10-(3′-Chloropropyl)-2-chloroacridone
10-(4′-N-Diethylaminobutyl)-2-chloroacridone
10-(4′-N-(Methylpiperazino) butyl)-2-chloroacridone
10-(4′-N-Piperidinobutyl)-2-chloroacridone
10-(4′-N-[(β-Hydroxyethyl)piperazino]butyl)-2-chloroacridone
10-[4′-N-Pyrrolidinobutyl]-2-chloroacridone
10-(4′-N-Morpholinobutyl)-2-chloroacridone
10-(4′-Chlorobutyl)-2-chloroacridone
10-(4′-N-Piperidinobutyl)-2-methoxyacridone
10-(4′-N-([β-Hydroxyethyl]piperazino)butyl)-2-bromoacridone
10-(3′-N-[(β-Hydroxyethyl) piperazino]propyl)-2-bromoacridone
10-(3′-N-[Bis[hydroxyethyl]amino]propyl)-2-bromoacridone
10-(4′-N-Chlorobutyl)-2-bromoacridone
10-(3′-N-Morpholinopropyl)-2-bromoacridone
10-(4′-[N-Diethylamino)butyl)-2-bromoacridone
10-(4′-N-Pyrrolidinobutyl)-2-bromoacridone
10-(4′-N-Morpholinobutyl)-2-bromoacridone
10-(3′-N-Piperidinopropyl)-2-bromoacridone
10-(4′-N-Thiomorpholinobutyl)-2-bromoacridone
10-(3′-N-Pyrrolidinopropyl)-2-bromoacridone
10-(3′-[N-Diethylamino]propyl)-2-bromoacridone
and pharmaceutically acceptable salts thereof.
